(19) United States
(12) Patent Application Publication
(10) Pub. No.: US 2026/0287596 A1
(43) Pub. Date: Sep. 24, 2026

(54) PROGNOSTIC AND PREDICTIVE VALUE OF ENDOTHELIAL DYSFUNCTION BIOMARKERS IN SEPSIS-ASSOCIATED ACUTE KIDNEY INJURY

(71) Applicant: CHILDREN'S HOSPITAL MEDICAL CENTER, Cincinnati, OH (US)

(72) Inventors: Mihir ATREYA, Cincinnati, OH (US); Natalja STANSKI, Cincinnati, OH (US)

(73) Assignee: CHILDREN'S HOSPITAL MEDICAL CENTER, Cincinnati, OH (US)

(21) Appl. No.: 19/168,598

(22) PCT Filed: May 10, 2024
(86) PCT No.: PCT/US2024/028949

§ 371 Date: Sep. 24, 2025

Related U.S. Application Data
(60) Provisional application No. 63/465,825, filed on May 11, 2023.

(51) Int. Cl. G01N 33/68; C12Q 1/6883; G16B 40/00; G16H 50/30

(52) U.S. Cl. CPC G01N 33/6893; C12Q 1/6883; G16B 40/00; G16H 50/30; C12Q 2600/118; C12Q 2600/158; G01N 2800/26; G01N 2800/347; G01N 2800/50

(57) ABSTRACT

Methods and compositions disclosed herein generally relate to methods of identifying, validating, and measuring clinically relevant, quantifiable biomarkers of diagnostic and therapeutic responses for blood, vascular, cardiac, and respiratory tract dysfunction, particularly as those responses relate to septic shock in pediatric patients. Certain aspects of the invention relate to identifying one or more biomarkers associated with septic shock in pediatric patients in combination with one or more endothelial-derived biomarkers, receiving a dataset comprising biomarker concentrations, wherein the dataset is from a sample obtained from a pediatric patient having at least one indication of septic shock, then determining whether the biomarker concentrations of each of the at least one biomarkers are greater than one or more pre-determined cut-off biomarker concentration, wherein the level of said biomarker correlates with a predicted outcome.

### CROSS REFERENCE TO RELATED APPLICATION

The present application claims the benefit of priority under 35 U.S.C. § 119(e) to U.S. Provisional Patent Application No. 63/465,825, PROGNOSTIC AND PREDICTIVE VALUE OF ENDOTHELIAL DYSFUNCTION BIOMARKERS IN SEPSIS-ASSOCIATED ACUTE KIDNEY INJURY: RISK STRATIFIED ANALYSIS FROM A PROSPECTIVE OBSERVATIONAL COHORT OF PEDIATRIC SEPTIC SHOCK, filed on filed May 11, 2023, which is currently co-pending herewith and which is incorporated by reference in its entirety.

### STATEMENT REGARDING FEDERALLY-SPONSORED RESEARCH

This invention was made with government support under Grant Nos. R35GM126943 and KL2TR001426 awarded by the National Institutes of Health. The government has certain rights in the invention.

### FIELD OF THE INVENTION

The invention disclosed herein generally relate to the identification and validation of clinically relevant, quantifiable biomarkers associated with sepsis and septic shock, and in more particular aspects to pediatric patients with sepsis-associated acute kidney injury.

### BACKGROUND

Sepsis-associated acute kidney injury (SA-AKI) is common among critically ill adults and children admitted to intensive care units (ICU), affecting up to half of patients with septic shock. SA-AKI is associated with high morbidity and mortality, with no current therapies available beyond continuous renal replacement therapy (CRRT). Systemic inflammation and microvascular endothelial dysfunction are key drivers of SA-AKI.

Given the substantial disease burden associated with SA-AKI, there remains a dire need to identify at-risk patients. In addition, it is particularly important to identify those patients who may have a biological predisposition to respond to targeted therapies.

### SUMMARY OF THE INVENTION

Various embodiments of the disclosure encompass methods of classifying a patient with septic shock as high risk of sepsis-associated acute kidney injury (SA-AKI, or SA-AKI SCr) or other than high risk of SA-AKI, the methods including: receiving a dataset comprising biomarker expression levels of one or more biomarkers selected from the group consisting of: Tie-2, Angpt-2, and sTM, wherein the dataset is obtained from a pediatric patient with septic shock at a first time point; determining whether the biomarker expression levels of each of the at least one biomarkers are greater than one or more pre-determined cut-off biomarker expression level; and classifying the patient as high risk of SA-AKI, or other than high risk of SA-AKI, based on the determination of whether the expression levels of each of the at least two biomarkers are greater than the one or more pre-determined cut-off expression level. In some embodiments, the biomarker expression levels are protein biomarker concentrations. In some embodiments, the protein biomarker concentrations are determined from a serum sample. In some embodiments, the dataset includes biomarker expression levels derived from a serum sample obtained from a pediatric patient with septic shock.

In some embodiments, a classification of high risk of SA-AKI includes: a) a non-highly elevated level of Tie-2, and absence of day 1 (D1) SA-AKI; b) a non-highly elevated level of Tie-2, presence of D1 SA-AKI, and an elevated Angpt-2/Tie-2 ratio; and a classification of other than high risk of SA-AKI includes: c) an elevated but non-highly elevated level of Tie-2, and absence of D1 SA-AKI; d) a non-highly elevated level of Tie-2, presence of day 1 D1 SA-AKI, and a non-elevated Angpt-2/Tie-2 ratio; or e) a highly elevated level of Tie-2.

In some embodiments, biomarker expression levels can be determined by, e.g., quantification of serum protein biomarker concentrations. In some embodiments, biomarker expression levels can be determined by, e.g., quantification of serum protein biomarker concentrations and/or by cycle threshold (CT) values.

In some embodiments, the determined biomarker expression levels include expression levels of one or more pairs of biomarkers selected from the group consisting of: Tie-2 and Angpt-2; Tie-2 and sTM; and Angpt-2 and sTM. In some embodiments, the determined biomarker expression levels include expression levels of Tie-2, Angpt-2, and sTM.

In some embodiments, biomarker levels are determined by serum protein biomarker concentration, wherein: a) an elevated level of Tie-2 corresponds to a serum Tie-2 concentration greater than 11.1 ng/ml; b) a highly elevated level of Tie-2 corresponds to a serum Tie-2 concentration greater than 28.6 ng/ml; c) an elevated Angpt-2/Tie-2 ratio corresponds to a ratio greater than 0.354753; and d) an elevated level of sTM corresponds to a serum sTM concentration greater than 11.8 ng/ml.

In some embodiments, the determination of whether the levels of the at least two biomarkers are non-elevated above a cut-off level includes applying the biomarker expression level data to a decision tree comprising the two or more biomarkers. In some embodiments, the decision tree of FIG. 5 can be applied. In some embodiments, a classification other than high risk includes a classification of low risk or intermediate risk.

In some embodiments, SA-AKI includes cardiovascular, respiratory, renal, hepatic, hematologic, and/or neurologic dysfunction, and/or systemic inflammation and/or microvascular endothelial dysfunction, and/or low or no urine output, fluid overload with edema, increased need for supplemental oxygen or intubation and mechanical ventilation, need for dialysis, multi-organ failure, and/or death. In some embodiments, SA-AKI includes renal dysfunction. In some embodiments, the patient can be undergoing continuous renal replacement therapy (CRRT).

In some embodiments, high risk of SA-AKI by day 3 of septic shock or other than high risk of SA-AKI by day 3 of septic shock can be determined. In some embodiments, high risk of SA-AKI by day 7 of septic shock or other than high risk of SA-AKI by day 7 of septic shock can be determined.

In some embodiments, the classification can be combined with one or more patient demographic data and/or clinical characteristics and/or results from other tests or indicia of septic shock and/or one or more additional biomarkers and/or platelet count. In some embodiments, the one or more additional biomarkers can be selected from: interleukin-8 (IL-8), heat shock protein 70 kDa 1B (HSPA1B), C—C Chemokine ligand 3 (CCL3), C—C Chemokine ligand 4 (CCL4), Granzyme B (GZMB), Interleukin-1 α (IL-1a), Matrix metallopeptidase 8 (MMP8), Angiopoietin-1 (Angpt-1), Inter-Cellular Adhesion Molecule-1 (ICAM-1), Vascular cell adhesion molecule-1 (VCAM-1), P-selectin, E-selectin, and Platelet and endothelial cell adhesion molecule-1 (PECAM-1). In some embodiments, the one or more additional biomarkers can be selected from: interleukin-8 (IL-8), heat shock protein 70 kDa 1B (HSPA1B), and C—C Chemokine ligand 3 (CCL3). In some embodiments, the patient demographic data and/or clinical characteristics and/or results from other tests or indicia of septic shock can include at least one of: the septic shock causative organism, the presence or absence or chronic disease, and/or the age, gender, race, ethnicity, and/or co-morbidities of the patient.

In some embodiments, the classification can be combined with one or more additional population-based risk scores. In some embodiments, the one or more population-based risk scores includes at least one of: Pediatric Sepsis Biomarker Risk Model (PERSEVERE), Pediatric Sepsis Biomarker Risk Model II (PERSEVERE II), Pediatric Risk of Mortality (PRISM), PRISM III, Pediatric Index of Mortality (PIM), and Pediatric Logistic Organ Dysfunction (PELOD). In some embodiments, the one or more population-based risk scores includes PERSEVERE II.

In some embodiments, the sample can be obtained within the first hour of presentation with septic shock. In some embodiments, the sample can be obtained within the first 24 hours of presentation with septic shock.

In some embodiments, a treatment including one or more high risk therapy can be administered to a patient that is classified as high risk, or a treatment excluding a high risk therapy can be administered to a patient that is not high risk, to provide a method of treating a pediatric patient with septic shock. In some embodiments, the one or more high risk therapy includes at least one of: biological and/or immune enhancing therapy, extracorporeal membrane oxygenation/life support, plasmapheresis, pulmonary artery catheterization, high volume continuous hemofiltration, adjuvant hemoperfusion, and/or plasma filtration and/or adsorption therapies. In some embodiments, the biological and/or immune enhancing therapy includes administration of recombinant human thrombomodulin, Angiopoietin-2 inhibitors, and/or Tie-2 agonists.

In some embodiments, the patient can be enrolled in a clinical trial. In some embodiments, the patient can be enrolled in a clinical trial and can be classified as high risk. In some embodiments, the method includes prognostic enrichment through enrollment of the high risk patient in the clinical trial. In some embodiments, a treatment including one or more high risk therapy can be administered to the patient in the clinical trial.

In some embodiments, the methods further include improving an outcome in a pediatric patient with septic shock. In some embodiments, the methods further include: receiving a dataset comprising expression levels of one or more biomarkers comprising Tie-2, Angpt-2, and/or sTM, wherein the dataset is obtained from a second sample from the treated patient at a second time point; analyzing the second sample to determine the expression levels of, determining whether the protein biomarker expression levels of each of the biomarkers are greater than one or more pre-determined cut-off protein biomarker expression level; classifying the patient as high risk of SA-AKI, or other than high risk of SA-AKI, based on the determination of whether the expression levels of each of the biomarkers are greater than the one or more pre-determined cut-off expression level; and maintaining the treatment being administered if the patient's high risk classification has not changed, or changing the treatment being administered if the patient's high risk classification has changed.

In some embodiments, the second time point can be at least 18 hours after the first time point. In some embodiments, the second time point can be in the range of 24 to 96 hours, or longer, after the first time point. In some embodiments, the second time point can be about 1 day, 2 days, 3 days, or longer, after the first time point. In some embodiments, the second time point can be about 2 days after the first time point. In some embodiments, the first time point can be at day 1, wherein day 1 can be within 24 hours of a septic shock diagnosis, and the second time point can be at day 3.

In some embodiments, a patient classified as high risk after the second time point can be administered one or more high risk therapy. In some embodiments, the one or more high risk therapy includes at least one selected from: biological and/or immune enhancing therapy, extracorporeal membrane oxygenation/life support, plasmapheresis, pulmonary artery catheterization, high volume continuous hemofiltration, adjuvant hemoperfusion, and/or plasma filtration and/or adsorption therapies. In some embodiments, the one or more high risk therapy includes a biological and/or immune enhancing therapy. In some embodiments, a patient not classified as high risk after the second time point can be administered a treatment excluding a high risk therapy. In some embodiments, the patient classified as high risk and administered one or more high risk therapy after the first time point is not classified as high risk after the second time point.

In some embodiments, the methods can be used as part of a companion diagnostic or a point of care device or kit.

In some embodiments, one or more biomarker cut-off level can be determined by one or more trained machine learning models based on a dataset generated from a cohort of pediatric patients with and without SA-AKI. In some embodiments, the data from the cohort of pediatric patients with and without SA-AKI can be provided to one or more machine learning models as input, and the one or more trained machine learning model can be based on a dataset generated from the biomarker cutoff levels in the patients of the cohort. In some embodiments, one or more biomarker cut-off level can be determined by a trained machine learning model, and one or more machine learning models can be used to classify the patient as high risk of SA-AKI, or other than high risk of SA-AKI.

Embodiments of the disclosure also encompass diagnostic kits, tests, arrays, and point of care devices including a reporter hybridization probe, and a capture hybridization probe specific for each of two or more mRNA, DNA, or protein biomarkers selected from: Tie-2, Angpt-2, and sTM. In some embodiments, the kit, test, array, and point of care biomarkers include Tie-2, Angpt-2, and sTM. In some embodiments, the kit, test, array, and point of care device biomarkers additionally include one or more of interleukin-8 (IL-8), heat shock protein 70 kDa 1B (HSPA1B), C—C Chemokine ligand 3 (CCL3), C—C Chemokine ligand 4 (CCL4), Granzyme B (GZMB), Interleukin-1 α (IL-1a), and/or Matrix metallopeptidase 8 (MMP8). In some embodiments, the kits, tests, arrays, and point of care devices additionally include a collection cartridge for immobilization of the hybridization probes. In some embodiments, the reporter and the capture hybridization probes include signal and barcode elements, respectively.

Embodiments of the disclosure also encompass apparatuses or processing devices suitable for detecting two or more biomarkers selected from: Tie-2, Angpt-2, and sTM. In some embodiments, the apparatus or processing device biomarkers include Tie-2, Angpt-2, and sTM. In some embodiments, the biomarkers additionally include one or more of interleukin-8 (IL-8), heat shock protein 70 kDa 1B (HSPA1B), C—C Chemokine ligand 3 (CCL3), C—C Chemokine ligand 4 (CCL4), Granzyme B (GZMB), Interleukin-1 α (IL-1a), and/or Matrix metallopeptidase 8 (MMP8).

Embodiments of the disclosure also encompass compositions including a reporter hybridization probe, and a capture hybridization probe specific for each of two or more biomarkers selected from: Tie-2, Angpt-2, and sTM. In some embodiments, the compositions include Tie-2, Angpt-2, and sTM. In some embodiments, the biomarkers further include one or more of interleukin-8 (IL-8), heat shock protein 70 kDa 1B (HSPA1B), C—C Chemokine ligand 3 (CCL3), C—C Chemokine ligand 4 (CCL4), Granzyme B (GZMB), Interleukin-1 α (IL-1a), and/or Matrix metallopeptidase 8 (MMP8).

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. An example computer system, upon which embodiments, or portions of the embodiments, may be implemented, in accordance with various embodiments.

FIG. 2. Exemplary flow diagram demonstrating inclusion and exclusion of patients in the cohort. Abbreviations: Serum Creatinine (SCr). Pediatric Sepsis Biomarker Risk Model (PERSEVERE-II).

FIG. 3. Exemplary box and whisker plots of concentrations (pg/mL) of endothelial dysfunction marker concentrations among patients with and without Day 3 sepsis-associated acute kidney injury based on serum creatinine (D3 SA-AKI SCr). Y axis is depicted in log scale. Asterisk * indicates a p value of 0.01. **** indicates a p value of <0.0001.

FIG. 4. Box and whisker plots of concentrations of endothelial dysfunction markers among an exemplary cohort of patients with and without Day 3 sepsis-associated acute kidney injury (D3 SA-AKI), across low-, intermediate-, and high PERSEVERE-II mortality risk strata. Asterisk indicates that the interaction between D3 SA-AKI and PERSEVERE-II mortality risk strata influenced concentrations of Tie-2 and Angpt-2/Tie-2 ratio.

FIG. 5. The PERSEVERANCE SA-AKI CART Model. Exemplary classification and regression analyses tree (CART) model to estimate risk of Day 3 sepsis-associated acute kidney injury based on serum creatinine criteria (D3 SA-AKI SCr) among patients with high- or intermediate-PERSEVERE-II mortality risk strata. Terminal nodes (TN) 1, 4, and 5 were deemed to have a high-risk of D3 SA-AKI (>71.4%); TN2, 3, and 6 were considered to have low-risk of D3 SA-AKI (<71.4%). The decision tree depicts Yes D3 SA-AKI SCr vs No D3 SA-AKI SCr.

FIG. 6. FIG. 6A illustrates the exemplary receiver operating characteristic curve for the exemplary PERSEVERENCE SA-AKI CART model to estimate risk of Day 3 sepsis-associated acute kidney injury (D3 SA-AKI) among patients with high- or intermediate-PERSEVERE-II mortality risk strata in training and test sets. FIG. 6B illustrates relative variable importance of predictor variables included in the exemplary model. Variable importance measures model improvement when splits are made on a predictor; relative importance is defined as % improvement with respect to the top predictor.

FIG. 7. Exemplary classification of patients with high- and intermediate-PERSEVERE-II mortality risk in the validation cohort (n=84) according to the presently derived PERSEVERENCE SA-AKI risk model without any modifications. The decision tree depicts Yes D3 SA-AKI SCr vs No D3 SA-AKI SCr.

### DETAILED DESCRIPTION OF THE INVENTION

All references cited herein are incorporated by reference in their entirety. Also incorporated herein by reference in their entirety include: U.S. Patent Application No. 61/595,996, BIOMARKERS OF SEPTIC SHOCK, filed on Feb. 7, 2012; U.S. Provisional Application No. 61/721,705, A MULTI-BIOMARKER-BASED OUTCOME RISK STRATIFICATION MODEL FOR ADULT SEPTIC SHOCK, filed on Nov. 2, 2012; International Patent Application No. PCT/US2013/25223, A MULTI-BIOMARKER-BASED OUTCOME RISK STRATIFICATION MODEL FOR PEDIATRIC SEPTIC SHOCK, filed on Feb. 7, 2013; International Patent Application No. PCT/US2013/25221, A MULTI-BIOMARKER-BASED OUTCOME RISK STRATIFICATION MODEL FOR ADULT SEPTIC SHOCK, filed on Feb. 7, 2013; U.S. Provisional Application No. 61/908,613, TEMPORAL PEDIATRIC SEPSIS BIOMARKER RISK MODEL, filed on Nov. 25, 2013; International Patent Application No. PCT/US2014/067438, TEMPORAL PEDIATRIC SEPSIS BIOMARKER RISK MODEL, filed on Nov. 25, 2014; U.S. patent application Ser. No. 15/998,427, SEPTIC SHOCK ENDOTYPING STRATEGY AND MORTALITY RISK FOR CLINICAL APPLICATION, filed on Aug. 15, 2018; U.S. Provisional Application No. 62/616,646, TEMPORAL ENDOTYPE TRANSITIONS REFLECT CHANGING RISK AND TREATMENT RESPONSE IN PEDIATRIC SEPTIC SHOCK, filed on Jan. 12, 2018; International Application No. PCT/US2017/032538, SIMPLIFICATION OF A SEPTIC SHOCK ENDOTYPING STRATEGY FOR CLINICAL APPLICATIONS, filed on May 12, 2017; U.S. Provisional Application No. 62/335,803, SIMPLIFICATION OF A SEPTIC SHOCK ENDOTYPING STRATEGY FOR CLINICAL APPLICATIONS, filed on May 13, 2016; U.S. Provisional Application No. 62/427,778, SIMPLIFICATION OF A SEPTIC SHOCK ENDOTYPING STRATEGY FOR CLINICAL APPLICATIONS, filed on Nov. 29, 2016; U.S. Provisional Application No. 62/428,451, SIMPLIFICATION OF A SEPTIC SHOCK ENDOTYPING STRATEGY FOR CLINICAL APPLICATIONS, filed on Nov. 30, 2016; U.S. Provisional Application No. 62/446,216, SIMPLIFICATION OF A SEPTIC SHOCK ENDOTYPING STRATEGY FOR CLINICAL APPLICATIONS, filed on Jan. 13, 2017; U.S. patent application Ser. No. 16/539,128, SEPTIC SHOCK ENDOTYPING STRATEGY AND MORTALITY RISK FOR CLINICAL APPLICATION, filed on Aug. 13, 2019; U.S. Provisional Application No. 62/764,831, Endotype Transitions During the Acute Phase of Pediatric Septic Shock Reflect Changing Risk and Treatment Response, filed on Aug. 15, 2018; U.S. Provisional Application No. 63/149,744, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2021; International Patent Application No. PCT/US2022/016642, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2022; U.S. Provisional Application No. 63/347,504, PREDICTING PERSISTENT MULTIPLE ORGAN DYSFUNCTION IN THE PEDIATRIC POPULATION AFTER CARDIOPULMONARY BYPASS USING SEPSIS PROGNOSTIC BIOMARKERS, filed on May 31, 2022; and U.S. Provisional Patent Application No. PEDIATRIC SEPSIS MULTIPLE ORGAN DYSFUNCTION SYNDROME RISK PREDICTION MODEL, filed on Jun. 1, 2022.

Unless otherwise noted, terms are to be understood according to conventional usage by those of ordinary skill in the relevant art.

As used herein, the term “sample” encompasses a sample obtained from a subject or patient. The sample can be of any biological tissue or fluid. Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, tear fluid, or cells therefrom. Samples can also include sections of tissues such as frozen or fixed sections taken for histological purposes or micro-dissected cells or extracellular parts thereof. A sample to be analyzed can be tissue material from a tissue biopsy obtained by aspiration or punch, excision or by any other surgical method leading to biopsy or resected cellular material. Such a sample can comprise cells obtained from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” includes any form of measurement, and includes determining if an element is present or not. The terms “determining,” “measuring,” “evaluating,” “assessing” and “assaying” can be used interchangeably and can include quantitative and/or qualitative determinations.

As used herein, the term “monitoring” with reference to septic shock refers to a method or process of determining the severity or degree of septic shock or stratifying septic shock based on risk and/or probability of mortality. In some embodiments, monitoring relates to a method or process of determining the therapeutic efficacy of a treatment being administered to a patient.

As used herein, “outcome” can refer to an outcome studied. In some embodiments in accordance with the present disclosure, “outcome” can refer to the presence of severe SA-AKI on day 3 of septic shock.

In some embodiments, “outcome” can include survival/mortality at day 7 and/or day 28. The importance of survival/mortality in the context of pediatric septic shock is readily evident. The common choice of 28 days was based on the fact that 28-day mortality is a standard primary endpoint for interventional clinical trials involving critically ill patients. In some embodiments, an increased risk for a poor outcome indicates that a therapy has had a poor efficacy, and a reduced risk for a poor outcome indicates that a therapy has had a good efficacy. In some embodiments, “outcome” can refer to resolution of organ failure after 14 days or 28 days or limb loss. Although mortality/survival is obviously an important outcome, survivors have clinically relevant short- and long-term morbidities that impact quality of life, which are not captured by the dichotomy of “alive” or “dead.” In the absence of a formal, validated quality of life measurement tool for survivors of pediatric septic shock, resolution of organ failure can be used as a secondary outcome measure. For example, the presence or absence of new organ failure over one or more timeframes can be tracked. Patients having organ failure beyond 28 days are likely to survive with significant morbidities having negative consequences for quality of life. Organ failure is generally defined based on published and well-accepted criteria for the pediatric population. Specifically, cardiovascular, respiratory, renal, hepatic, hematologic, and neurologic failure can be tracked. In addition, limb loss can be tracked as a secondary outcome. Although limb loss is not a true “organ failure,” it is an important consequence of pediatric septic shock with obvious impact on quality of life.

In some embodiments, “outcome” can refer to organ dysfunction and/or death after septic shock. In some embodiments, “outcome” can refer to two or more organ dysfunctions or death by day 7 of septic shock. In some embodiments, “outcome” can refer to day 7 cardiovascular, respiratory, renal, hepatic, hematologic, and neurologic dysfunction.

In some embodiments, “outcome” can include complicated course. Complicated course as defined herein relates to persistence of two or more organ failures at day seven of septic shock or 28-day mortality; this can be a composite of death during the study period or the persistence of 2 or more organ dysfunctions on day 7 of septic shock, PICU length of stay (LOS), and/or PIC.

As used herein, the terms “predicting outcome” and “outcome risk stratification” with reference to septic shock refers to a method or process of prognosticating a patient's risk of a certain outcome. In some embodiments, predicting an outcome relates to monitoring the therapeutic efficacy of a treatment being administered to a patient. In some embodiments, predicting an outcome relates to determining a relative risk of an adverse outcome (e.g. complicated course) and/or mortality. In some embodiments, the predicted outcome is associated with administration of a particular treatment or treatment regimen. Such adverse outcome risk and/or mortality can be high risk, moderate risk, moderate-high risk, moderate-low risk, or low risk. Alternatively, such adverse outcome risk can be described simply as high risk or low risk, corresponding to high risk of adverse outcome (e.g. complicated course) and/or mortality probability, or high likelihood of therapeutic effectiveness, respectively. In some embodiments of the present invention, adverse outcome risk can be determined via the biomarker-based SA-AKI risk stratification as described herein. In some embodiments, predicting an outcome relates to determining a relative risk of SA-AKI. Such mortality risk can be high risk, moderate risk, moderate-high risk, moderate-low risk, or low risk. Alternatively, such mortality risk can be described simply as high risk or low risk, corresponding to high risk of death or high likelihood of survival, respectively. As related to the terminal nodes of the decision trees described herein, a “high risk terminal node” corresponds to an increased probability of adverse outcome (e.g. complicated course) and/or mortality according to a particular treatment or treatment regimen, whereas a “low risk terminal node” corresponds to a decreased probability of adverse outcome (e.g. complicated course) and/or mortality according to a particular treatment or treatment regimen.

As used herein, the term “high risk clinical trial” refers to one in which the test agent has “more than minimal risk” (as defined by the terminology used by institutional review boards, or IRBs). In some embodiments, a high risk clinical trial is a drug trial.

As used herein, the term “low risk clinical trial” refers to one in which the test agent has “minimal risk” (as defined by the terminology used by IRBs). In some embodiments, a low risk clinical trial is one that is not a drug trial. In some embodiments, a low risk clinical trial is one that that involves the use of a monitor or clinical practice process. In some embodiments, a low risk clinical trial is an observational clinical trial.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” refers to any member of the animal kingdom. In some embodiments, a subject is a human patient. In some embodiments, a subject is a pediatric patient. In some embodiments, a pediatric patient is a patient under 18 years of age, while an adult patient is 18 or older. Unless stated otherwise, the terms “patient” or “child” (or “patients” or “children”) refer to a pediatric patient (i.e., under 18 years old).

As used herein, the terms “treatment,” “treating,” “treat,” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition.

As used herein, the term “marker” or “biomarker” refers to a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, the term “expression levels” refers, for example, to a determined level of biomarker expression. The term “pattern of expression levels” refers to a determined level of biomarker expression compared either to a reference (e.g. a housekeeping gene or inversely regulated genes, or other reference biomarker) or to a computed average expression value (e.g. in DNA-chip analyses). A pattern is not limited to the comparison of two biomarkers but is more related to multiple comparisons of biomarkers to reference biomarkers or samples. A certain “pattern of expression levels” can also result and be determined by comparison and measurement of several biomarkers as disclosed herein and display the relative abundance of these transcripts to each other.

As used herein, a “reference pattern of expression levels” refers to any pattern of expression levels that can be used for the comparison to another pattern of expression levels. In some embodiments of the invention, a reference pattern of expression levels is, for example, an average pattern of expression levels observed in a group of healthy or diseased individuals, serving as a reference group.

As used herein, the term “decision tree” refers to a standard machine learning technique for multivariate data analysis and classification. Decision trees can be used to derive easily interpretable and intuitive rules for decision support systems.

Persistent sepsis-associated acute kidney injury (SA-AKI) is associated with high mortality rates, ranging from 30 to 70%, and new functional morbidity among affected patients. Unfortunately, there are no disease modifying therapies available for SA-AKI, forcing clinicians to rely solely on supportive care measures such as continuous renal replacement therapy (CRRT) to treat affected patients.

Dysregulated systemic inflammatory response to infection is undoubtedly a key contributor to the development of SA-AKI. In children specifically, it has been previously demonstrated that the updated Pediatric Sepsis Biomarker Risk Model (PERSEVERE-II, a prospectively validated prognostic enrichment tool to estimate mortality risk that incorporates pediatric sepsis-specific inflammatory biomarkers and platelet count) is highly predictive of severe SA-AKI and its sequelae. Of note, the incidence of SA-AKI and use of CRRT were higher, while renal recovery was lower, among children with high- or intermediate-PERSEVERE-II mortality strata relative to those with low-risk.

Microcirculatory changes, including increased vascular permeability with interstitial edema and resultant tubular epithelial hypoxia, are thought to be a pathognomonic feature of SA-AKI. Translational studies among critically ill adults corroborate this important role of endothelial dysfunction in SA-AKI. More recently, studies evaluating subphenotypes of SA-AKI in critically ill adults have demonstrated worse outcomes among those with both systemic inflammation and endothelial dysfunction, with subphenotype assignment carrying prognostic and potential therapeutic implications. However, such data are lacking among critically ill children, who may manifest age-related differences in sepsis pathobiology.

Accordingly, the association between markers of endothelial dysfunction and Day 3 (D3) SA-AKI was assessed among children with septic shock. Further, analyses were conducted to test whether the association between endothelial biomarkers and Day 3 (D3) SA-AKI risk differed across PERSEVERE-II mortality risk strata. Finally, endothelial biomarker-based models were derived and validated to estimate the risk of D3 SA-AKI among prespecified subgroups, based on PERSEVERE-II mortality risk.

### Determining Risk of SA-AKI

As described herein, the present inventors measured differences between endothelial dysfunction markers among children with and without SA-AKI, tested whether this association varied across inflammatory biomarker-based risk strata (e.g. morbidity and mortality risk strata), and developed prediction models to identify those at highest risk of sepsis associated acute kidney injury (SA-AKI).

The study used secondary analyses of a prospective observational cohort of pediatric septic shock. The primary outcome of interest was the presence of >Stage II Kidney Disease Improving Global Outcomes (KDIGO) SA-AKI on day 3 based on serum creatinine (D3 SA-AKI SCr, also referred to herein as D3 SA-AKI SCr). Biomarkers including those prospectively validated to predict pediatric sepsis mortality (PERSEVERE-II) were measured in day 1 (D1) serum. Multivariable regression was used to test the independent association between endothelial markers and D3 SA-AKI SCr. Risk-stratified analyses were conducted, and prediction models were developed, using Classification and Regression Tree (CART), to estimate risk of D3 SA-AKI among prespecified subgroups based on PERSEVERE-II risk.

A total of 414 patients were included in the derivation cohort. Patients with D3 SA-AKI SCr had worse clinical outcomes including 28-day mortality and need for CRRT. Serum soluble thrombomodulin (sTM), Angiopoietin-2 (Angpt-2), and Tie-2 were independently associated with D3 SA-AKI SCr. Further, Tie-2 and Angpt-2/Tie-2 ratios were influenced by the interaction between D3 SA-AKI SCr and risk strata. Logistic regression demonstrated models predictive of D3 SA-AKI risk performed optimally among patients with high- or intermediate-PERSEVERE-II risk strata. A 6 terminal node CART model restricted to this subgroup of patients had an area under the receiver operating characteristic curve (AUROC) 0.90 and 0.77 upon tenfold cross-validation in the derivation cohort to distinguish those with and without D3 SA-AKI SCr and high specificity. The newly derived model performed well in a unique set of patients (n=224), 84 of whom were deemed high- or intermediate-PERSEVERE-II risk, to distinguish those patients with high vs. low risk of D3 SA-AKI SCr.

Thus, endothelial dysfunction biomarkers were found to be independently associated with risk of severe SA-AKI. Incorporation of endothelial biomarkers can thus facilitate prognostic and predictive enrichment for selection of therapeutics in future clinical trials among critically ill children.

As described herein, an association was discovered between markers of endothelial dysfunction and severe SA-AKI in a large cohort of critically ill children with septic shock. In the cohort described in the Examples, sTM, Angpt-2, and Tie-2 were independently associated with increased odds of D3 SA-AKI SCr. Further, endothelial dysfunction markers tested demonstrated variable response across PERSEVERE-II mortality risk strata, with Tie-2 concentrations and Angpt-2/Tie-2 ratios being influenced by the interaction between D3 SA-AKI SCr and PERSEVERE-II mortality risk strata. Subsequently, the performance of an endothelial biomarker-based model was derived and validated. This model predicted with high specificity the risk of D3 SA-AKI SCr among patients with a high- or intermediate-PERSEVERE-II mortality risk.

Several studies have reported on the association between endothelial dysfunction markers and risk of SA-AKI among adults. Single-center studies have identified the independent association of sTM, Angpt-2, and Angpt-1 with risk of AKI among critically ill adults, a vast majority of whom had sepsis as the inciting cause. Similar observations have been made among septic patients enrolled in the multi-center Finnish Acute Kidney Injury (FINNAKI) cohort, where both sTM and Angpt-2 were associated with an independent risk of 90-day mortality, and more recently among patients with severe sepsis and acute respiratory failure enrolled in the Validating Acute Lung Injury markers for Diagnosis (VALID) study where Angpt-2 outperformed other biomarkers in predicting risk of SA-AKI. Importantly, the time point at which AKI was determined ranged from >12 hours up to 7 days from the time of ICU admission.

The present data among critically ill children corroborate those among adults. There is a major distinction, however, in that the present analysis focuses on D3 SA-AKI SCr, a clinically significant time point beyond which spontaneous recovery of kidney function is less likely and as such associated with poor outcomes.

Recently, Bhatraju et al. (Am J Respir Crit Care Med 199, 863-872 (2019)) and Wiersma et al. (Crit Care 24, 150 (2020)) have identified subphenotypes of SA-AKI among critically ill adults with phenotypes AKI-SP2 and Subphenotype 2, respectively, demonstrating high levels of systemic inflammation and endothelial activation. In particular, AKI-SP2 was characterized by a high Angpt-2/Angpt-1 ratio—a key variable that helped distinguish subphenotypes. Accordingly, Angpt-2/Angpt-1, which serve to regulate microvascular barrier integrity, could be linked to development of SA-AKI. The present data that low Tie-2 and high Angpt-2/Tie-2 ratios are highly predictive of D3 SA-AKI SCr among the most critically ill subset of pediatric septic shock, categorized as high- or intermediate-PERSEVERE-II mortality risk, are both novel and complementary to the previous findings, which relate to adults.

The results described herein can thus inform prognostic enrichment efforts to identify those at highest risk of SA-AKI among critically ill patients with septic shock. Indeed, several of the endothelial biomarkers including sTM, Angpt-2, Tie-2, and Angpt-2/Tie-2 ratio were among the top predictor variables predictive of SA-AKI on day 7 in studies by the present group that sought to develop models that have integrated PERSEVERE-endothelial biomarkers to develop a unified model to predict risk of multiple organ dysfunctions. Such integrated models or alternatively sequential deployment of PERSEVERE-II followed by the endothelial biomarker-based risk models in real time can allow for identification of high-risk patients who may be amenable to targeted therapies.

The PERSEVERANCE SA-AKI CART prediction model detailed in this study has biologic plausibility. Tie-2 is an important molecule that plays a key role in stabilizing the endothelial barrier integrity and preventing capillary leak. Angpt-2 antagonizes the effect of Tie-2 and serves to disrupt its function. Finally, thrombomodulin plays a vital role to inhibit coagulation pathway by serving as a co-factor in thrombin mediated activation of protein C. However, it has been demonstrated that Angpt-2 also binds and inhibits thrombomodulin function. In concordance with these biological roles, patients with higher Tie-2 concentrations in the cohort described herein had a low-risk of D3 SA-AKI SCr. Moreover, those with high Angpt-2/Tie-2 ratios and soluble thrombomodulin had a high-risk of D3 SA-AKI SCr. It is conceivable that those patient with high Angpt-2/Tie-2 ratios had higher sTM concentrations in response to elevated Angpt-2.

A randomized trial of recombinant human soluble thrombomodulin (rhTM) among 800 adult patients with sepsis-associated coagulopathy failed to demonstrate a 28-day all-cause mortality among critically ill patients. More recently, in a retrospective single center study in Japan among 97 adult patients with SA-AKI, rhTM administration was associated with lower 28-day mortality, improvement in renal function, and reduced use of renal replacement therapy at ICU discharge. It remains plausible that the subset of patients identified by the PERSEVERANCE SA-AKI prediction model can have a biological predilection to respond to microvascular stabilizing therapies, including rhTM, that seek to restore the balance between Angpt-2/Tie-2 and improve endothelial barrier function. The present model can therefore be used to facilitate predictive enrichment in future clinical trials of such therapies among critically ill children with high risk of persistent and severe AKI. These data can dramatically inform future translational approaches to improving the care of critically ill children with high risk of severe SA-AKI.

These data can be used to inform future translational approaches to improving the care of critically ill children with SA-AKI. The study described herein is particularly strong, given that it included a large cohort of pediatric septic shock patients. Also, unlike other black box machine learning algorithms, CART methodology provides clear biomarker thresholds based on which patients are divided into risk strata and can be used to test model performance in other populations. In addition, fivefold cross-validation and internal validation of the model were performed in a unique set of patients. These data represent real-world challenges to model performance when using biomarkers, including the potential for batch-to-batch variation in measurements.

Biomarkers of endothelial dysfunction are independently associated with risk of severe sepsis-associated kidney injury and demonstrate variable responses across pediatric sepsis mortality (e.g. PERSEVERE-II) risk strata. The risk prediction models developed herein can facilitate prognostic and predictive enrichment of critically ill children for selection in precision microvascular stabilizing therapies, which can meaningfully improve SA-AKI outcomes and treatment selection.

### Additional Patient Information

The demographic data, clinical characteristics, and/or results from other tests or indicia of septic shock specific to a pediatric patient with septic shock can affect the patient's outcome risk. Accordingly, such demographic data, clinical characteristics, and/or results from other tests or indicia of septic shock can be incorporated into the methods described herein which allow for stratification of individual pediatric patients in order to determine the patient's outcome risk. Such demographic data, clinical characteristics, and/or results from other tests or indicia of septic shock can also be used in combination with the methods described herein which allow for stratification of individual pediatric patients in order to determine the patient's outcome risk.

Such pediatric patient demographic data can include, for example, the patient's age, race, ethnicity, gender, and the like. In some embodiments, the biomarker-based SA-AKI risk stratification described herein can incorporate or be used in combination with the patient's age, race, ethnicity, and/or gender to determine an outcome risk.

Such patient clinical characteristics and/or results from other tests or indicia of septic shock can include, for example, the patient's co-morbidities and/or septic shock causative organism, and the like.

Patient co-morbidities can include, for example, acute lymphocytic leukemia, acute myeloid leukemia, aplastic anemia, atrial and ventricular septal defects, bone marrow transplantation, caustic ingestion, chronic granulomatous disease, chronic hepatic failure, chronic lung disease, chronic lymphopenia, chronic obstructive pulmonary disease (COPD), congestive heart failure (NYHA Class IV CHF), Cri du Chat syndrome, cyclic neutropenia, developmental delay, diabetes, DiGeorge syndrome, Down syndrome, drowning, end stage renal disease, glycogen storage disease type 1, hematologic or metastatic solid organ malignancy, hemophagocytic lymphohistiocytosis, hepatoblastoma, heterotaxy, hydrocephalus, hypoplastic left heart syndrome, IPEX Syndrome, kidney transplant, Langerhans cell histiocytosis, liver and bowel transplant, liver failure, liver transplant, medulloblastoma, metaleukodystrophy, mitochondrial disorder, multiple congenital anomalies, multi-visceral transplant, nephrotic syndrome, neuroblastoma, neuromuscular disorder, obstructed pulmonary veins, Pallister Killian syndrome, Prader-Willi syndrome, requirement for chronic dialysis, requirement for chronic steroids, retinoblastoma, rhabdomyosarcoma, rhabdosarcoma, sarcoma, seizure disorder, severe combined immune deficiency, short gut syndrome, sickle cell disease, sleep apnea, small bowel transplant, subglottic stenosis, tracheal stenosis, traumatic brain injury, trisomy 18, type 1 diabetes mellitus, unspecified brain tumor, unspecified congenital heart disease, unspecified leukemia, VATER Syndrome, Wilms tumor, and the like. Any one or more of the above patient co-morbidities can be indicative of the presence or absence of chronic disease in the patient.

Septic shock causative organisms can include, for example, Acinetobacter baumannii, Adenovirus, Bacteroides species, Candida species, Capnotyophaga jenuni, Cytomegalovirus, Enterobacter cloacae, Enterococcus faecalis, Escherichia coli, Herpes simplex virus, Human metapneumovirus, Influenza A, Klebsiella pneumonia, Micrococcus species, mixed bacterial infection, Moraxella catarrhalis, Neisseria meningitides, Parainfluenza, Pseudomonas species, Serratia marcescens, Staphylococcus aureus, Streptococcus agalactiae, Streptococcus milleri, Streptococcus pneumonia, Streptococcus pyogenes, unspecified gram negative rods, unspecified gram positive cocci, and the like.

In some embodiments, the biomarker-based SA-AKI SCr risk stratification as described herein can incorporate the patient's co-morbidities to determine an outcome risk and/or mortality probability. In some embodiments, the biomarker-based SA-AKI SCr risk stratification as described herein can incorporate the patient's septic shock causative organism to determine an outcome risk and/or mortality probability.

In some embodiments, the biomarker-based SA-AKI SCr risk stratification as described herein can be used in combination with the patient's co-morbidities to determine an outcome risk and/or mortality probability. In some embodiments, the biomarker-based SA-AKI SCr risk stratification as described herein can be used in combination with the patient's septic shock causative organism to determine an outcome risk and/or mortality probability.

Certain embodiments of the invention include using quantification data from a gene-expression analysis and/or from a protein, mRNA, and/or DNA analysis, from a sample of blood, urine, saliva, broncho-alveolar lavage fluid, or the like. Embodiments of the invention include not only methods of conducting and interpreting such tests but also include reagents, compositions, kits, tests, arrays, apparatuses, processing devices, assays, and the like, for conducting the tests. The compositions and kits of the present invention can include one or more components which enable detection of the biomarkers disclosed herein and combinations thereof and can include, but are not limited to, primers, probes, cDNA, enzymes, covalently attached reporter molecules, and the like.

Diagnostic-testing procedure performance is commonly described by evaluating control groups to obtain four critical test characteristics, namely positive predictive value (PPV), negative predictive value (NPV), sensitivity, and specificity, which provide information regarding the effectiveness of the test. The PPV of a particular diagnostic test represents the proportion of positive tests in subjects with the condition of interest (i.e. proportion of true positives); for tests with a high PPV, a positive test indicates the presence of the condition in question. The NPV of a particular diagnostic test represents the proportion of negative tests in subjects without the condition of interest (i.e. proportion of true negatives); for tests with a high NPV, a negative test indicates the absence of the condition. Sensitivity represents the proportion of subjects with the condition of interest who will have a positive test; for tests with high sensitivity, a positive test indicates the presence of the condition in question. Specificity represents the proportion of subjects without the condition of interest who will have a negative test; for tests with high specificity, a negative test indicates the absence of the condition.

The threshold for the disease state can alternatively be defined as a 1-D quantitative score, or diagnostic cutoff, based upon receiver operating characteristic (ROC) analysis. The quantitative score based upon ROC analysis can be used to determine the specificity and/or the sensitivity of a given diagnosis based upon subjecting a patient to a decision tree described herein in order to predict an outcome for a pediatric patient with septic shock.

The correlations disclosed herein, between pediatric patient septic shock biomarker levels and/or mRNA levels and/or gene expression levels, and/or protein expression levels, provide a basis for conducting a diagnosis of septic shock, or for conducting a stratification of patients with septic shock, or for enhancing the reliability of a diagnosis of septic shock by combining the results of a quantification of a septic shock biomarker with results from other tests or indicia of septic shock, or for determining an appropriate treatment regimen for a pediatric patient with septic shock. For example, the results of a quantification of one biomarker could be combined with the results of a quantification of one or more additional biomarker, protein, cytokine, mRNA, or the like. Thus, even in situations in which a given biomarker correlates only moderately or weakly with septic shock, providing only a relatively small PPV, NPV, specificity, and/or sensitivity, the correlation can be one indicium, combinable with one or more others that, in combination, provide an enhanced clarity and certainty of diagnosis. Accordingly, the methods and materials of the invention are expressly contemplated to be used both alone and in combination with other tests and indicia, whether quantitative or qualitative in nature.

### PERSEVERE, PERSEVERE II, and Other Population-Based Risk Scores

As mentioned previously, the PERSEVERE model for estimating baseline mortality risk in children with septic shock was previously derived and validated. PERSEVERE is based on a panel of 12 serum protein biomarkers measured from blood samples obtained during the first 24 hours of a septic shock diagnosis, selected from among 80 genes having an association with mortality risk in pediatric septic shock. Of those 12 serum biomarkers, the derived and validated PERSEVERE model is based on Interleukin-8 (IL-8), Heat shock protein 70 kDA (HSP70), C—C Chemokine ligand 3 (CCL3), C—C Chemokine ligand 4 (CCL4), Granzyme B (GZMB), Interleukin-1 α (IL-1a), and Matrix metallopeptidase 8 (MMP8). PERSEVERE additionally takes patient age into account.

The PERSEVERE decision tree has 8 terminal nodes. Of these, 3 terminal nodes of the PERSEVERE decision tree are determined to be low risk/low mortality probability (terminal nodes 2, 4, and 7), while 5 terminal nodes of the PERSEVERE decision tree are determined to be intermediate to high risk/high mortality probability (terminal nodes 1, 3, 5, 6, and 8). In some embodiments, a low risk/low mortality probability terminal nodes has a mortality probability between 0.000 and 0.025, while an intermediate to high risk/high mortality probability terminal nodes has a mortality probability greater than 0.025.

In some embodiments of the present invention, a patient sample is analyzed for the PERSEVERE serum protein biomarkers IL-8 and HSP70, as well as for the endothelial biomarkers ICAM-1, Thrombomodulin, Angpt-2/Angpt-1, and/or Angpt-2/Tie-2.

In some embodiments of the present invention, the PERSEVERE mortality probability stratification can be used in combination with the biomarker-based SA-AKI risk stratification as described herein. In some embodiments, the biomarker-based SA-AKI risk stratification, as described herein, can be used in combination with a patient endotyping strategy and/or Z score determination. In some embodiments, the combination of a biomarker-based SA-AKI risk stratification, with an endotyping strategy and/or Z score determination, can be used to determine an appropriate treatment regimen for a patient. For example, such combinations can be used to identify which patients are more likely to benefit from corticosteroids.

As mentioned previously, the PERSEVERE II model for estimating baseline mortality risk in children with septic shock was previously derived and validated. PERSEVERE II is based on a panel of 5 serum protein biomarkers measured from blood samples obtained during the first 24 hours of a septic shock diagnosis. Of those 5 serum biomarkers, the derived and validated PERSEVERE II model is based on interleukin-8 (IL-8), C—C chemokine ligand 3 (CCL3), and heat shock protein 70 kDa 1B (HSPA1B), as well as platelet count.

The PERSEVERE II decision tree has 5 terminal nodes. Of these, 3 terminal nodes of the PERSEVERE II decision tree are determined to be low risk/low mortality probability (terminal nodes 1, 2, and 4), while 2 terminal nodes of the PERSEVERE II decision tree are determined to be intermediate to high risk/high mortality probability (terminal nodes 3 and 5). In some embodiments, a low risk/low mortality probability terminal nodes has a mortality probability between 0.000 and 0.025, while an intermediate to high risk/high mortality probability terminal nodes has a mortality probability greater than 0.025.

In some embodiments of the present invention, a patient sample is analyzed for the PERSEVERE II serum protein biomarkers IL-8, CCL3, and HSPA1B, and platelet count, as well as for the endothelial biomarkers Tie-2, Angpt-2, and sTM.

In some embodiments of the present invention, the PERSEVERE II mortality probability stratification can be used in combination with the biomarker-based SA-AKI risk stratification as described herein. In some embodiments, the biomarker-based SA-AKI risk stratification, as described herein, can be used in combination with a patient endotyping strategy and/or Z score determination. In some embodiments, the combination of a biomarker-based SA-AKI risk stratification, with an endotyping strategy and/or Z score determination, can be used to determine an appropriate treatment regimen for a patient. For example, such combinations can be used to identify which patients are more likely to benefit from corticosteroids.

A number of additional models that generate mortality prediction scores based on physiological variables have been developed to date. These can include the PRISM, Pediatric Index of Mortality (PIM), and/pediatric logistic organ dysfunction (PELOD) models, and the like.

Such models can be very effective for estimating population-based outcome risks but are not intended for stratification of individual patients. The methods described herein which allow for stratification of individual patients can be used alone or in combination with one or more existing population-based risk scores.

In some embodiments, the biomarker-based SA-AKI risk stratification described herein can be used with one or more additional population-based risk scores. In some embodiments, the biomarker-based SA-AKI risk stratification described herein can be used in combination with PRISM. In some embodiments, the biomarker-based SA-AKI risk stratification described herein can be used in combination with PIM. In some embodiments, the biomarker-based SA-AKI risk stratification herein can be used in combination with PELOD. In some embodiments, the biomarker-based SA-AKI risk stratification described herein can be used in combination with a population-based risk score other than PRISM, PIM, and PELOD.

### High Risk Therapies

High risk, invasive therapeutic and support modalities can be used to treat septic shock. The methods described herein which allow for the patient's outcome risk to be determined can help inform clinical decisions regarding the application of high risk therapies to specific pediatric patients, based on the patient's outcome risk.

High risk therapies include, for example, adjuvant hemoperfusion, plasma filtration and adsorption therapies, extracorporeal membrane oxygenation/life support, plasmapheresis, pulmonary artery catheterization, high volume continuous hemofiltration, and the like. High risk therapies can also include non-corticosteroid therapies, e.g. alternative therapies and/or high risk therapies. In particular, patients at high risk of SA-AKI can be treated with immune enhancing therapies, such as, for example, recombinant human thrombomodulin, Angiopoietin-2 inhibitors, Tie-2 agonists, and the like.

In some embodiments, individualized treatment can be provided to a pediatric patient by selecting a pediatric patient classified as high risk by the methods described herein for one or more high risk therapies. In some embodiments, individualized treatment can be provided to a pediatric patient by excluding a pediatric patient classified as low risk from one or more high risk therapies.

### Computer Implemented System

In various embodiments, the systems and methods for recognizing home activities by deep learning subtle vibrations on an interior surface of a house from a single point using vibration sensing devices can be implemented via computer software or hardware. Refer to the Appendix for further information regarding the system, devices and methods provided herein, in accordance with various embodiments.

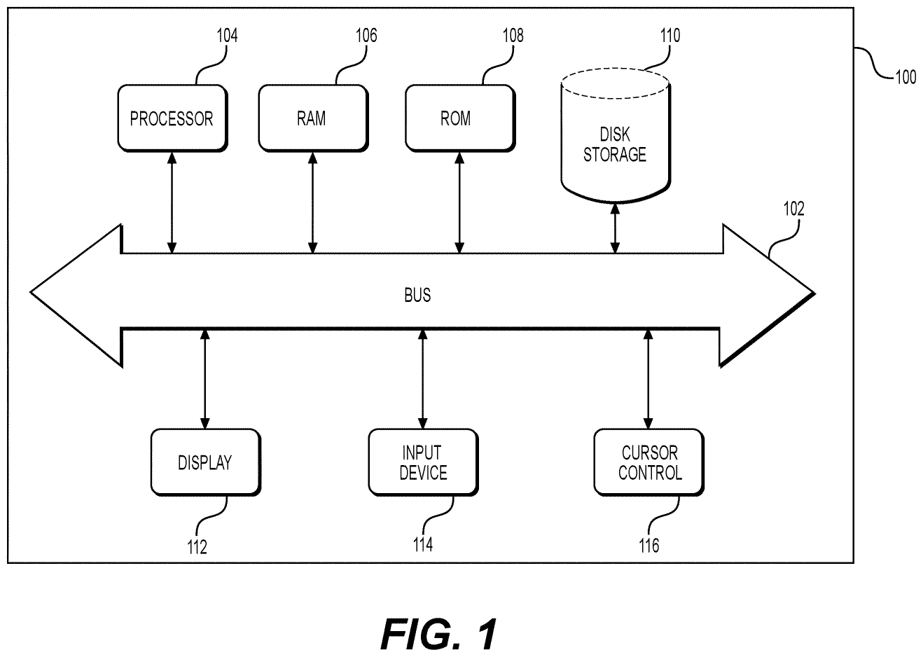

FIG. 1 is a block diagram illustrating a computer system 100 upon which embodiments of the present teachings may be implemented. In various embodiments of the present teachings, computer system 100 can include a bus 102 or other communication mechanism for communicating information and a processor 104 coupled with bus 102 for processing information. In various embodiments, computer system 100 can also include a memory, which can be a random-access memory (RAM) 106 or other dynamic storage device, coupled to bus 102 for determining instructions to be executed by processor 104. Memory can also be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communication of information and command selections to processor 104. Another type of user input device is a cursor control 116, such as a mouse, a trackball or cursor direction keys for communicating direction information and command selections to processor 104 and for controlling cursor movement on display 112. This input device 114 typically has two degrees of freedom in two axes, a first axis (i.e., x) and a second axis (i.e., y), that allows the device to specify positions in a plane. However, it should be understood that input devices 114 allowing for 3-dimensional (x, y and z) cursor movement are also contemplated herein.

Consistent with certain implementations of the present teachings, results can be provided by computer system 100 in response to processor 104 executing one or more sequences of one or more instructions contained in memory 106. Such instructions can be read into memory 106 from another computer-readable medium or computer-readable storage medium, such as storage device 110. Execution of the sequences of instructions contained in memory 106 can cause processor 104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 104 for execution. Such a medium can take many forms, including but not limited to, non-volatile media, volatile media, and transmission media. Examples of non-volatile media can include, but are not limited to, dynamic memory, such as memory 106. Examples of transmission media can include, but are not limited to, coaxial cables, copper wire, and fiber optics, including the wires that comprise bus 10

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 104 of computer system 100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 100 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 100, whereby processor 104 would execute the analyses and determinations provided by these engines, subject to instructions provided by any one of, or a combination of, memory components 106/108/110 and user input provided via input device 114.

In various embodiments, the methods of the present teachings can involve deep learning and/or machine learning and/or one or more neural network, such as a deep neural network, and the like. It should be understood that while deep learning and such processes may be discussed in conjunction with various embodiments herein, the various embodiments herein are not limited to being associated only with deep learning tools. As such, machine learning and/or artificial intelligence tools generally may be applicable as well. Moreover, the terms deep learning, machine learning, and artificial intelligence may even be used interchangeably in generally describing the various embodiments of systems, software and methods herein.

A deep neural network (DNN) generally, such as a convolutional neural network (CNN), generally accomplishes an advanced form of image processing and classification/detection by first looking for low level features such as, for example, edges and curves, and then advancing to more abstract (e.g., unique to the type of images being classified) concepts through a series of convolutional layers. A DNN/CNN can do this by passing an image through a series of convolutional, nonlinear, pooling (or downsampling, as will be discussed in more detail below), and fully connected layers, and get an output. Again, the output can be a single class or a probability of classes that best describes the image or detects objects on the image.

Regarding layers in a CNN, for example, the first layer is generally a convolutional layer (Conv). This first layer will process the image's representative array using a series of parameters. Rather than processing the image as a whole, a CNN will analyze a collection of image sub-sets using a filter (or neuron or kernel). The sub-sets will include a focal point in the array as well surrounding points. For example, a filter can examine a series of 5×5 areas (or regions) in a 32×32 image. These regions can be referred to as receptive fields. Since the filter must possess the same depth of the input, an image with dimensions of 32×32×3 would have a filter of the same depth (e.g., 5×5×3). The actual step of convolving, using the exemplary dimensions above, would involve sliding the filter along the input image, multiplying filter values with the original pixel values of the image to compute element wise multiplications, and summing these values to arrive at a single number for that examined portion of the image.

After completion of this convolving step, using a 5×5×3 filter, an activation map (or filter map) having dimensions of 28×28×1 will result. For each additional layer used, spatial dimensions are better preserved such that using two filters will result in an activation map of 28×28×2. Each filter will generally have a unique feature it represents (e.g., colors, edges, curves, etc.) that, together, represent the feature identifiers required for the final image output. These filters, when used in combination, allow the CNN to process an image input to detect those features present at each pixel. Therefore, if a filter serves as a curve detector, the convolving of the filter along the image input will produce an array of numbers in the activation map that correspond to high likelihood of a curve (high summed element wise multiplications), low likelihood of a curve (low summed element wise multiplications) or a zero value where the input volume at certain points provided nothing that would activate the curve detector filter. As such, the greater number of filters (also referred to as channels) in the Cony, the more depth (or data) that is provided on the activation map, and therefore more information about the input that will lead to a more accurate output.

Balanced with accuracy of the CNN is the processing time and power needed to produce a result. In other words, the more filters (or channels) used, the more time and processing power needed to execute the Conv. Therefore, the choice and number of filters (or channels) to meet the needs of the CNN method are specifically chosen to produce as accurate an output as possible while considering the time and power available.

To enable further a CNN to detect more complex features, additional Conv layers can be added to analyze what outputs from the previous Conv layer (i.e., activation maps). For example, if a first Conv layers looks for a basic feature such as a curve or an edge, a second Conv layer can look for a more complex feature such as shapes, which can be a combination of individual features detected in an earlier Conv layer. By providing a series of Conv layers, the CNN can detect increasingly higher-level features to arrive eventually at the specific desired object detection. Moreover, as the Conv layers stack on top of each other, analyzing the previous activation map output, each Conv layer in the stack is naturally going to analyze a larger and larger receptive field by virtue of the scaling down that occurs at each Conv level, thereby allowing the CNN to respond to a growing region of pixel space in detecting the object of interest.

A CNN architecture generally consists of a group of processing blocks, including at least one processing block for convoluting an input volume (image) and at least one for deconvolution block (or transpose convolution). Additionally, the processing blocks can include at least one pooling block and unpooling block. Pooling blocks can be used to scale down an image in resolution to produce an output available for Conv. This can provide computational efficiency (efficient time and power), which can in turn improve actual performance of the CNN. Those these pooling, or subsampling, blocks keep filters small and computational requirements reasonable, these blocks coarsen the output (can result in lost spatial information within a receptive field), reducing it from the size of the input by a factor equal to the pixel stride of the receptive fields of the output units.

Unpooling blocks can be used to reconstruct a these coarse outputs to produce an output volume with the same dimensions as the input volume. An unpooling block can be considered a reverse operation of a convoluting block to return an activation output to the original input volume dimension.

However, the unpooling process generally just simply enlarges the coarse outputs into a sparse activation map. To avoid this result, the deconvolution block densifies this sparse activation map to produce both and enlarged and dense activation map that eventually, after any further necessary processing, a final output volume with size and density much closer to the input volume. As a reverse operation of the convolution block, rather than reducing multiple array points in the receptive field to a single number, the deconvolution block associate a single activation output point with a multiple outputs to enlarge and densify the resulting activation output.

It should be noted that while pooling blocks can be used to scale down an image and unpooling blocks can be used to enlarge these scaled down activation maps, convolution and deconvolution blocks can be structured to both convolve/deconvolve and scale down/enlarge without separate pooling and unpooling blocks.

The pooling and unpooling process can be limited depending on the objects of interest being detected in an image input. Since pooling generally scales down an image by looking at sub-image windows without overlap of windows, there is a clear loss in spatial info as the scaling down occurs.

A processing block can include other layers that are packaged with a convolutional or deconvolutional layer. These can include, for example, a rectified linear unit layer (ReLU) or exponential linear unit layer (ELU), which are activation functions that examine the output from a Conv layer in its processing block. The ReLU or ELU layer acts as a gating function to advance only those values corresponding to positive detection of the feature of interest unique to the Conv layer its processing block.

Given a basic architecture, the CNN is then prepared for a training process to hone its accuracy in image classification/detection (of objects of interest). Using training data sets, or sample images used to train the CNN so that it updates its parameters in reaching an optimal, or threshold, accuracy, a process called backpropagation (backprop) occurs. Backpropagation involves a series of repeated steps (training iterations) that, depending on the parameters of the backprop, either will slowly or quickly train the CNN. Backprop steps generally include forward pass, loss function, backward pass, and parameter (weight) update according to a given learning rate. The forward pass involves passing a training image through the CNN. The loss function is a measure of error in the output. The backward pass determines the contributing factors to the loss function. The weight update involves updating the parameters of the filters to move the CNN towards optimal. The learning rate determines the extent of weight update per iteration to arrive at optimal. If the learning rate is too low, the training may take too long and involve too much processing capacity. If the learning rate is too fast, each weight update may be too large to allow for precise achievement of a given optimum or threshold.

The backprop process can cause complications in training, thus leading to the desire for lower learning rates and more specific and carefully determined initial parameters upon start of training. One such complication is that, as weight updates occur at the conclusion of each iteration, the changes to the parameters of the Conv layers amplify the deeper the network goes. For example, if a CNN has a plurality of Conv layers that, as discussed above, allows for higher-level feature analysis, the parameter update to the first Conv layer is multiplied at each subsequent Conv layer. The net effect is that the smallest changes to parameters have large impact depending on the depth of a given CNN. This phenomenon is referred to as internal covariate shift.

It should be noted that even though CNNs are spoken about in detail above, the various embodiments discussed herein could utilize any neural network type or architecture.

Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Methods

### Study Design and Patient Selection:

The study protocol was approved by Institutional Review Boards of participating institutions. Briefly, patients under the age of 18 years were recruited from multiple pediatric ICUs (PICU) across the USA between 2003 and 2019. There were no study-related interventions except for blood draws. Clinical and laboratory data were available between Day 1 through 7, including platelet counts on Day 1 (D1). Baseline illness severity among patients was determined by pediatric risk of mortality (PRISM-III) score.

Inclusion criteria for this study were: 1) patients meeting pediatric-specific consensus criteria for septic shock.

Exclusion criteria included: (1) patients with pre-existing kidney disease (n=60); (2) lack of serum creatinine (SCr) data on day 3 of septic shock (n=229); and (3) those with no endothelial dysfunction marker data on D1 (n=483).

Severe SA-AKI was defined using serum creatinine (SCr) criteria as per Kidney Disease Improving Global Outcomes (KDIGO) stage 2 AKI or higher, which corresponds to a >twofold increase in SCr relative to baseline. Baseline SCr values were unknown for all patients in the cohort, and thus were imputed using their calculated body surface area (m2) and an eGFR of 120 ml/min per 1.73 m2, as has been validated in the literature. Urine output data were not available for all patients in the cohort and therefore not included. All patients receiving RRT were considered to have severe AKI.

The primary outcome of interest was the presence of severe SA-AKI, based on serum creatinine criteria alone, on day 3 of septic shock, which is a clinically relevant time point in AKI research. The outcome is henceforth annotated as Day 3 SA-AKI SCr. Secondary outcomes included mortality at day 7 and day 28, complicated course (a composite of death during study period or the persistence of 2 or more organ dysfunctions on day 7 of septic shock), PICU length of stay (LOS), PICU free days (calculated by subtracting PICU LOS from a theoretical maximum of 28 days), and the use of CRRT.

### Persevere-II Based Risk Stratification:

PERSEVERE-II mortality probability and risk strata were determined, according to published methods (Crit Care Med 44, 2010-2017 (2016)). Briefly, Interleukin-8 (IL-8), Heat shock protein 70 kDA (HSP70), C—C Chemokine ligand 3 (CCL3), C—C Chemokine ligand 4 (CCL4), Granzyme B (GZMB), Interleukin-1a (IL-1a), and Matrix metallopeptidase 8 (MMP8) were previously measured in serum collected on D1.

Classification and Regression Tree (CART) analyses were used to derive a mortality probability risk score (0.000-0.999) using R software (version 4.2.2). Patients were subsequently classified as low risk (mortality probability score range <0.019), intermediate risk (mortality probability score range >0.019 to <0.300), or high risk (mortality probability score range >0.300).

### Serum Biomarkers of Endothelial Dysfunction:

Concentrations (in pg/mL) of soluble thrombomodulin (sTM), Angiopoietin-1 (Angpt-1), Angiopoietin-2 (Angpt-2), tyrosine kinase with immunoglobulin-like loops and epidermal growth factor homology domains-2 (Tie-2), intercellular adhesion molecule-1 (ICAM-1), Vascular Cell Adhesion Molecule-1 (VCAM-1), and Platelet Endothelial Cell Adhesion Molecule (PECAM-1) were measured in serum collected on day 1 of septic shock by Luminex assays (R&D Systems, MN).

### Statistical Analyses:

Minitab Software (PA, USA, version 21.1.0) was used for data analyses. GraphPad Prism (CA, USA, version 9) was used to generate figures. Demographic data were summarized with percentages or median with interquartile ranges. Differences between groups were determined by x test for categorical variables and by nonparametric Kruskal-Wallis H test for continuous variables. One-way analysis of variance (ANOVA) with Dunnett's test for correction for multiple comparisons was used when comparing differences across mortality risk strata.

Univariate and multivariable logistic regression with backward elimination of predictor variables (α<0.05) was used to test the association between predictor variables and risk of D3 SA-AKI SCr. The latter model was adjusted for patient age, PRISM-III score, and PERSEVERE-II mortality probability score. General linear models were used to test the variation in endothelial dysfunction markers according to D3 SA-AKI SCr status, PERSEVERE-II mortality class, and an interaction term for D3 SA-AKI SCr X PERSEVERE-II mortality class. A p value of 0.05 was used to test for statistical significance.

### Risk Prediction Modeling:

Multivariable logistic regression models incorporating biomarkers selected in the previous steps and presence of severe SA-AKI on day 1 (D1 SA-AKI SCr) were used to predict risk of D3 SA-AKI SCr across the entire cohort, and predefined subgroups including: (1) a composite of PERSEVERE-II high- and intermediate-mortality risk strata; and (2) PERSEVERE-II low-risk mortality risk strata alone. These subgroups were defined a priori as the number of patients deemed to be high- or intermediate-mortality risk was expected to be relatively low. Area under the receiver operating characteristic curve (AUROC) and diagnostic test characteristics are presented for training and fivefold cross-validation in the derivation cohort.

A new CART model was subsequently derived to optimize risk prediction among the subset of patients with high- or intermediate-PERSEVERE-II mortality risk using published approaches (Critical Care 26, 210 (2022)). Briefly, models were weighted to match sample frequencies and within K=1 standard error of minimum misclassification cost was chosen to select the optimal tree. Class probability method and tenfold cross validation was used for CART analyses. Patients were categorized as high- versus low-D3 SA-AKI SCr risk categories based on output of the model.

### Validation in a Unique Set of Patients:

The independent performance of the newly derived risk model, henceforth referred to as PERSEVER-ENCE SA-AKI model, was then tested in a unique set of patients with existing PERSEVERE-II biomarker data and newly measured endothelial markers. The presence of D3 SA-AKI SCr was compared among patients categorized as having high- versus low-risk of D3 SA-AKI SCr in the hold-out validation cohort using x test. The R code for the model can be provided on request and for the purposes of external validation.

### Example 2

### Patient Cohorts

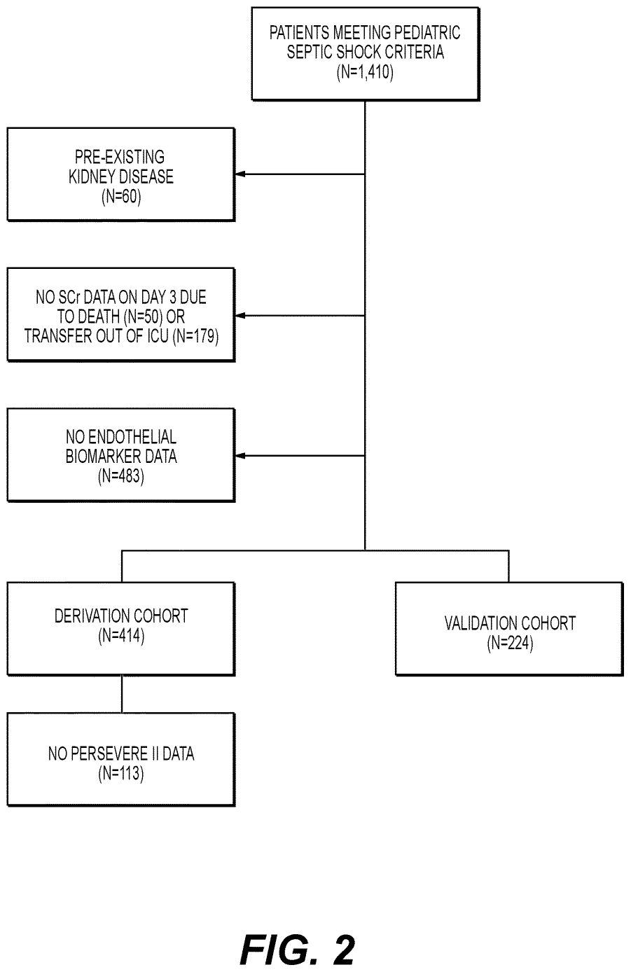

A total of 414 patients with pediatric septic shock were included in the derivation cohort and 224 patients were included in the validation cohort, as shown in FIG. 2. One hundred and forty patients (33.8%) and 74 patients (33.0%) had D3 SA-AKI SCr in the derivation and validation cohorts, respectively.

The demographic, clinical characteristics, and outcomes of patients with and without D3 SA-AKI SCr in the derivation cohort are shown in Table 1. Patients who had D3 SA-AKI SCr were younger and had higher illness severity on day 1 of septic shock. All secondary outcomes including mortality, complicated course, and need for CRRT on day 7 were higher among those with D3 SA-AKI SCr.

A total of 301 patients in the derivation cohort had PERSEVERE-II biomarker data to estimate mortality probability and categorize patients according to mortality risk strata. Differences in clinical outcomes across low-, intermediate-, and high-risk PERSEVERE-II strata are presented in Table 2.

TABLE 1
Demographic data and clinical characteristics in the derivation
cohort among patients with and without D3 SA-AKI.

| | D3 SA-AKI | No D3 | |
|---|---|---|---|
| | SCr | SA-AKI SCr | |
| Variable | (n = 140) | (n = 274) | p value |
| Age (Years) | 2.4 (0.9, 6.7) | 4.1 (1.7, 7.7) | 0.003 |
| Sex, Female (%) | 59 (42.2%) | 131 (47.8%) | 0.274 |
| Race (Self-identified) | | | |
| White/Caucasian | 102 (72.9%) | 203 (74.1%) | 0.947 |
| Black/African American | 18 (12.9%) | 35 (12.8%) | |
| Other | 20 (14.2%) | 36 (13.1%) | |
| Ethnicity | | | |
| Hispanic or Latino | 25(17.9%) | 43 (15.7%) | 0.485 |
| PRISM-III | 15 (9, 21) | 11 (6, 15) | <0.001 |
| 28-day Mortality | 28 (20.0%) | 7 (2.6%) | <0.001 |
| Complicated course | 94 (67.2%) | 55 (20.1%) | <0.001 |
| PICU LOS | 11 (7, 20) | 7(3, 12) | <0.001 |
| PICU Free Days | 17 (8, 21) | 21 (16, 25) | <0.001 |
| Day 7 CRRT* | 29/120 (24.2%) | 1/267 (0.01%) | <0.001 |

*Adjusted for mortality prior to day 7.

TABLE 2
Clinical characteristics of patients according
to PERSEVERE-II mortality class.

| | High risk | Intermediate | Low risk | P |
|---|---|---|---|---|
| N = 301 | (n = 36) | risk (n = 63) | (n = 202) | value |
| Age (years) | 2.4 (1.1, 5.2) | 3.2 (0.7, 7.3) | 3.3 (1.2, 6.8) | 0.977 |
| PRISM-III | 22 (16, 28) * | 14 (10, 18) * | 10 (5, 14) | <0.001 |
| P-ll Mort. Prob | 0.398 (0.391, | 0.187 (0.181, | 0.009 (0.005, | <0.001 |
| | 0.407) ** | 0.193)* | 0.012) | |
| 28-day mortality | 11 (30.6%) ** | 9(14.3%)* | 3(1.5%) | <0.001 |
| Complicated course | 23 (63.9%) ** | 30 (47.6%) * | 60 (29.7%) | <0.001 |
| D1 SA-AKI | 25 (69.4%) ″ | 37 (58.7%) * | 64 (31.8%) | <0.001 |
| D3 SA-AKI | 21 (58.3%) ** | 29 (46.1%)* | 56 (27.7%) | <0.001 |
| D7 SA-AKI | 19(52.8%)** | 22 (34.9%) * | 45 (22.3%) | <0.001 |
| Day 1 RRT | 9 (25.0%) ** | 6 (9.5%) * | 4 (2.0%) | <0.001 |
| Day 3 RRT | 13 (36.1%)** | 9(14.3%)* | 7 (3.5%) | <0.001 |
| Day 7 RRT | 17(47.2%)** | 11 (17.5%)* | 9 (4.5%) | <0.001 |

* Indicates significant difference relative to low-risk strata alone after adjusting for multiple comparisons.
** Indicates significant difference relative to intermediate- and low-risk strata after adjusting multiple comparisons.

### Example 3

### Independent Association of Markers of Endothelial Dysfunction with Risk of D3 SA-AKI SCr

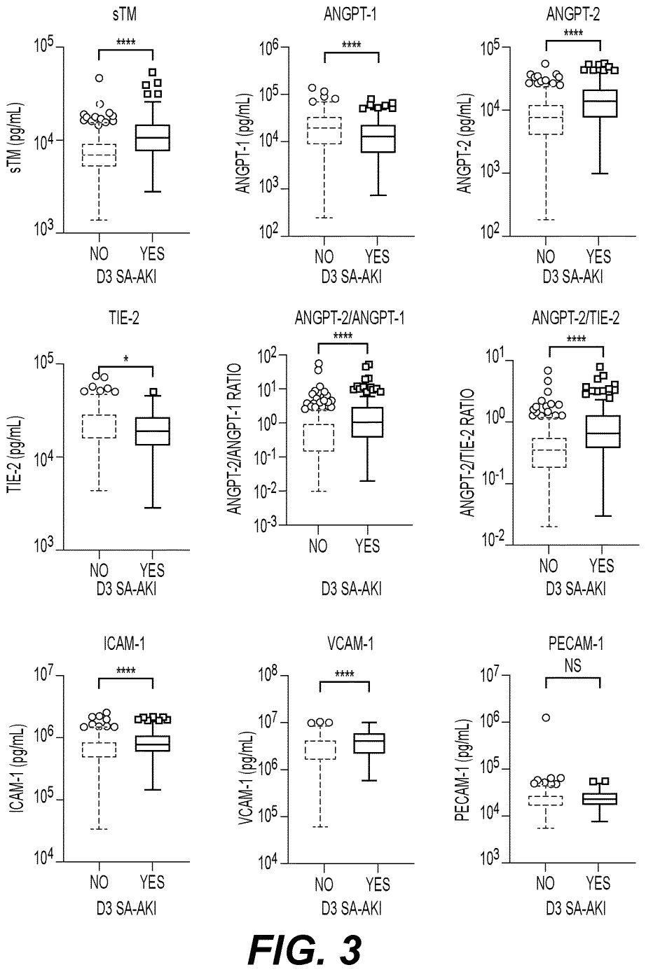

FIG. 3 shows markers of endothelial dysfunction among those with and without D3 SA-AKI SCr in the derivation cohort. Concentrations of all endothelial markers tested, except PECAM-1, differed between comparison groups of interest; sTM, Angpt-2, Angpt-2/Angpt-1 ratio, Angpt-2/Tie-2 ratio, VCAM-1, and ICAM-1 were higher; Angpt-1 and Tie-2 were lower among those with D3 SA-AKI SCr relative to those without. The univariate associations between predictor variables and the risk of D3 SA-AKI are shown in Table 3.

The results of multivariable logistic regression analyses to test the association between markers of endothelial dysfunction (log 10 transformed) and risk of D3 SA-AKI SCr are presented in Table 4. Only sTM, Angpt-2, and Tie-2 were independently associated with risk of D3 SA-AKI SCr. The adjusted odds of D3 SA-AKI SCr was 18.7 (95% CI: 3.2, 93.2) for each log 10 fold increase in sTM, 2.81 (95% CI: 1.1, 7.2) for each log 10 fold increase in Angpt-2, and 0.20 (95% CI: 0.05, 0.77) for each log 10 fold increase in Tie-2.

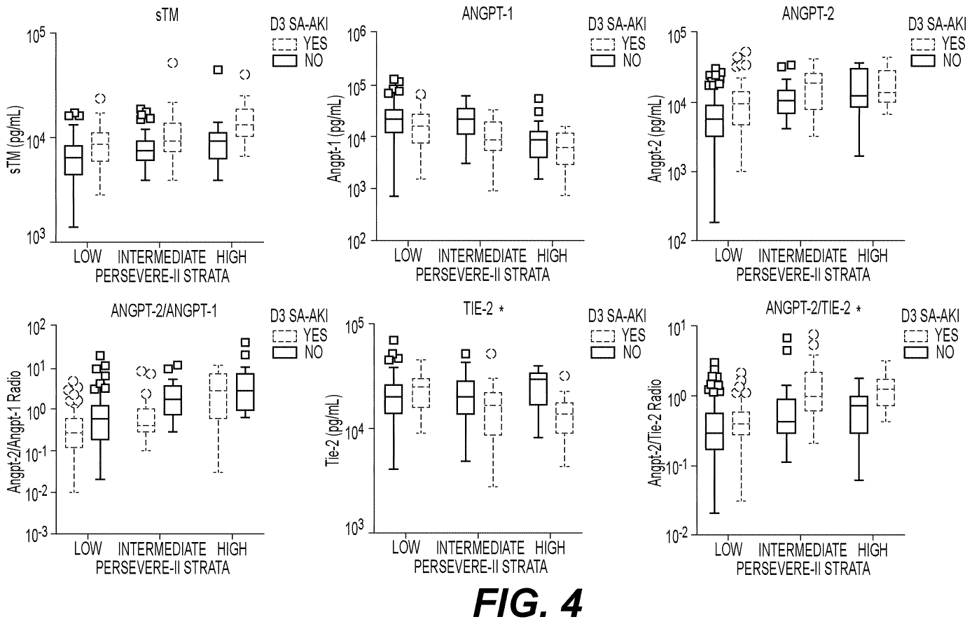

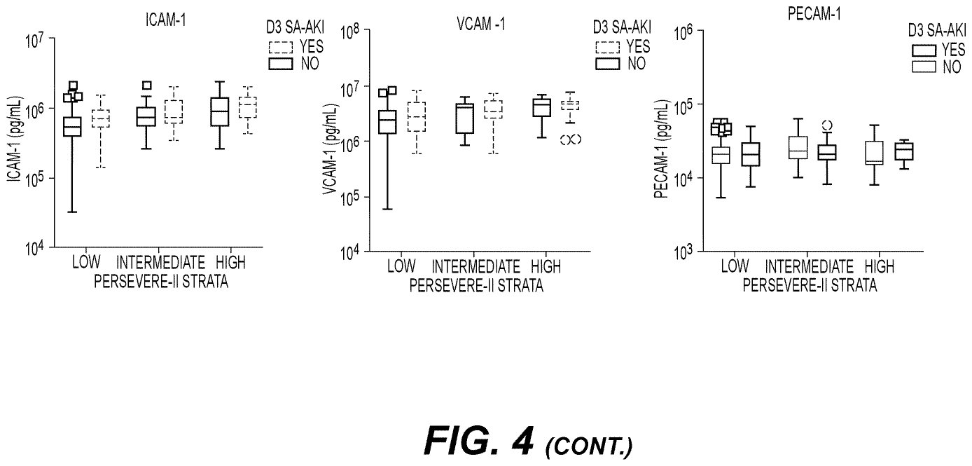

The association between markers of endothelial dysfunction and presence of D3 SA-AKI SCr varied across PERSEVERE-II mortality risk strata, as shown in FIG. 4 and Table 5. Of note, Tie-2 concentrations and Angpt-2/Tie-2 ratios were influenced by the interaction between the presence of D3 SA-AKI SCr and high-and intermediate-mortality risk strata.

TABLE 3
Univariate associations between predictor variables a
nd risk of D3 SA-AKI SCr.

| Variable | OR | P value |
|---|---|---|
| Age | 0.94 (0.90, 0.99) | 0.019 |
| Sex (Female) | 0.8 (0.5, 1.2) | 0.274 |
| PRISM-III | 1.04(1.01, 1.06) | 0.001 |
| P-ll Mortality Probability X10 | 1.4 (1.12, 1.7) | <0.001 |
| sTM (Log 10) | 56.4(18.4, 173.2) | <0.001 |
| Angpt-1 (Log10) | 0.4 (0.2, 0.6) | <0.001 |
| Angpt-2 (Log 10) | 7.4 (3.7, 14.3) | <0.001 |
| Tie-2 (Log 10) | 0.2 (0.1, 0.6) | 0.003 |
| Angpt-2/Angpt-1 | 1.07 (1.01, 1.14) | 0.015 |
| Angpt-2/Tie-2 | 2.6 (1.8, 3.7) | <0.001 |
| VCAM-1 (Log10) | 3.9 (2.0, 7.9) | <0.001 |
| ICAM-1 (Log10) | 14.7 (4.8, 43.7) | <0.001 |
| PECAM-1 (Log10) | 1.6 (0.6, 4.1) | 0.343 |

TABLE 4
Multi-variable logistic regression analysis to test the association
between D3 SA-AKI SCr and endothelial dysfunction markers in
the derivation cohort.

| | Coefficients | | Adj Odds | |
|---|---|---|---|---|
| Term | (SE) | p Value | Ratio | AUROC |
| Age (in years) | −0.04 (0.03) | 0.139 | 0.95 (0.89, 1.01) | 0.76 |
| PRISM-III | 0.04 (0.02) | 0.022 | 1.04 (1.01, 1.07) | |
| PERSEVERE-II | −0.02 (0.12) | 0.842 | 0.97 (0.77, 1.23) | |
| mortality probability* | | | | |
| sTM (Log10) | 2.93 (0.82) | <0.001 | 18.7 (3.8, 93.2) | |
| Angpt-2 (Log 10) | 1.03 (0.48) | 0.022 | 2.8 (1.1, 7.2) | |
| Tie-2 (Log 10) | −1.60 (0.68) | 0.019 | 0.20 (0.05, 0.77) | |

All endothelial dysfunction markers were considered in this model and backward elimination with an alpha of 0.05 was used to select variables
AUROC, Area under the receiver operating characteristic curve
*The raw PERSEVERE-II mortality probability was transformed by a factor of 10 for logistic regression analyses.

TABLE 5
General linear models for association between markers of endothelial
dysfunction, D3 SA-AKI SCr and PERSEVERE-II strata.

| Biomarker | Term | Coeff. (SE) | P value |
|---|---|---|---|
| sTM (Iog10) | D3 SA-AKI | 0.129 (0.03) | <0.001 |
| | P-ll Strata | | |
| | High | 0.169 (0.06) | 0.003 |
| | Intermediate | 0.116 (0.04) | 0.003 |
| | D3 SA-AKI X P-ll Strata | | |
| | D3 SA-AKI and High | 0.064 (0.07) | 0.402 |
| | D3 SA-AKI and Intermediate | −0.023 (0.06) | 0.698 |
| Angpt-1 (Iog10) | D3 SA-AKI | −0.118 (0.06) | 0.049 |
| | P-ll Strata | | |
| | High | −0.468 (0.11) | <0.001 |
| | Intermediate | −0.003 (0.07) | 0.966 |
| | D3 SA-AKI X P-ll Strata | | |
| | D3 SA-AKI and High | 0.066 (0.14) | 0.646 |
| | D3 SA-AKI and Intermediate | −0.185 (0.11) | 0.106 |
| Angpt-2 (log 10) | D3 SA-AKI | 0.204 (0.05) | <0.001 |
| | P-ll Strata | | |
| | High | 0.375 (0.09) | <0.001 |
| | Intermediate | 0.285 (0.06) | <0.001 |
| | D3 SA-AKI X P-ll Strata | | |
| | D3 SA-AKI and High | −0.098 (0.13) | 0.441 |
| | D3 SA-AKI and Intermediate | −0.036 (0.10) | 0.721 |
| Tie-2 (log10) | D3 SA-AKI | 0.054 (0.03) | 0.089 |
| | P-ll Strata | | |
| | High | 0.086 (0.05) | 0.117 |
| | Intermediate | 0.013 (0.04) | 0.730 |
| | D3 SA-AKI X P-ll Strata | | |
| | D3 SA-AKI and High | −0.311 (0.07) | <0.001 |
| | D3 SA-AKI and Intermediate | −0.215 (0.06) | <0.001 |
| Angpt-2/Angpt-1 | D3 SA-AKI | 1.037 (0.52) | 0.047 |
| | P-ll Strata | | |
| | High | 3.499 (0.92) | <0.001 |
| | Intermediate | 0.521 (0.63) | 0.413 |
| | D3 SA-AKI X P-Il Strata | | |
| | D3 SA-AKI and High | 1.090 (1.25) | 0.383 |
| | D3 SA-AKI and Intermediate | 0.389 (0.99) | 0.694 |
| Angpt-2/Tie-2 | D3 SA-AKI | 0.147 (0.12) | 0.238 |
| | P-ll Strata | | |
| | High | 0.322 (0.22) | 0.132 |
| | Intermediate | 0.463 (0.15) | 0.002 |
| | D3 SA-AKI X P-1I Strata | | |
| | D3 SA-AKI and High | 0.657 (0.30) | 0.027 |
| | D3 SA-AKI and Intermediate | 0.664 (0.23) | 0.005 |
| VCAM-1 | D3 SA-AKI | 0.08 (0.05) | 0.070 |
| | P-l I Strata | | |
| | High | 0.257 (0.08) | 0.002 |
| | Intermediate | 0.093 (0.05) | 0.102 |
| | D3 SA-AKI X P-ll Strata | | |
| | D3 SA-AKI and High | −0.072 (0.12) | 0.523 |
| | D3 SA-AKI and Intermediate | 0.008 (0.08) | 0.929 |
| ICAM-1 | D3 SA-AKI | 0.116 (0.03) | 0.001 |
| | P-ll Strata | | |
| | High | 0.202 (0.05) | 0.001 |
| | Intermediate | 0.144 (0.04) | 0.001 |
| | D3 SA-AKI X P-ll Strata | | |
| | D3 SA-AKI and High | −0.04 (0.08) | 0.622 |
| | D3 SA-AKI and Intermediate | −0.09 (0.06) | 0.129 |
| PECAM-1 | D3 SA-AKI | 0.013 (0.06) | 0.701 |
| | P-ll Strata | | |
| | High | 0.009 (0.06) | 0.874 |
| | Intermediate | 0.086 (0.04) | 0.043 |
| | D3 SA-AKI X P-ll Strata | | |
| | D3 SA-AKI and High | 0.017 (0.08) | 0.838 |
| | D3 SA-AKI and Intermediate | −0.06 (0.06) | 0.292 |

### Example 4

### Endothelial Dysfunction Marker-Based D3 SA-AKI SCr Risk Prediction Models Perform Better Among Patients Belonging to High- or Intermediate-PERSEVERE-II Mortality Risk Strata

The performance characteristics of multivariable logistic regression models predictive of D3 SA-AKI SCr which included the presence of severe D1 SA-AKI, sTM, Angpt-2, Tie-2, and Angpt-2/Tie-2 ratio in the training and cross-validation test sets are presented in Table 6. The AUROC for this model was 0.78 in the training set and 0.77 upon fivefold cross-validation across the entire cohort.

When restricted to those with high- or intermediate-PERSEVERE-II mortality risk, the model had better performance with AUROCs of 0.88 and 0.85 upon cross-validation, with sensitivity of 77.1% and 72.9% and specificity of 81.6% in both training and test sets. In comparison, the AUROCs were 0.77 and 0.73 among patients with low-mortality risk. In the low PERSEVERE-II mortality risk group, the model had high specificity but low sensitivity.

TABLE 6
Test characteristics of endothelial dysfunction marker-based
multivariable logistic regression model to estimate risk of
D3 SA-AKI SCr in the entire cohort and across predefined
groups based on PERSEVERE-II mortality risk.

| | |
|---|---|
| Training Set | Test Set |

Entire Cohort

| | | |
|---|---|---|
| AUROC | 0.78 | 0.77 |
| True positive, n | 52 | 51 |
| False negative, n | 51 | |
| False positive, n | 29 | 30 |
| True negative, n | 165 | 164 |
| Sensitivity % | 50.5% | 49.5% |
| Specificity % | 85.1% | 84.5% |
| False Positive Rate (%) | 14.9% | 15.5% |
| False Negative Rate | 49.5% | 50.5% |

High and Intermediate Risk P-ll Strata

| | | |
|---|---|---|
| AU ROC | 0.88 | 0.85 |
| True positive, n | 37 | 35 |
| False negative, n | 11 | 13 |
| False positive, n | 9 | 9 |
| True negative, n | 40 | 40 |
| Sensitivity % | 77.1% | 72.9% |
| Specificity % | 81.6% | 81.6% |
| False Positive Rate (%) | 18.4% | 18.4% |
| False Negative Rate (%) | 22.9% | 27.1% |

Low-Risk-P-ll Strata

| | | |
|---|---|---|
| AU ROC | 0.77 | 0.73 |
| True positive, n | 16 | 17 |
| False negative, n | 39 | 38 |
| False positive, n | 10 | 14 |
| True negative, n | 135 | 131 |
| Sensitivity % | 29.1% | 30.9% |
| Specificity % | 93.1% | 90.3% |
| False Positive Rate (%) | 6.9% | 9.7% |
| False Negative Rate (%) | 70.9% | 69.1% |

Incorporated D1 SA-AKI, sTM, Angpt-2, Tie-2, and Angpt-2/Tie-2 ratio.

### Example 5

### Classification and Regression Tree Analyses Yield an Optimal Model to Predict D3 SA-AKI Risk Among Subset of Patients with High- or Intermediate-PERSEVERE-II Mortality Risk Strata

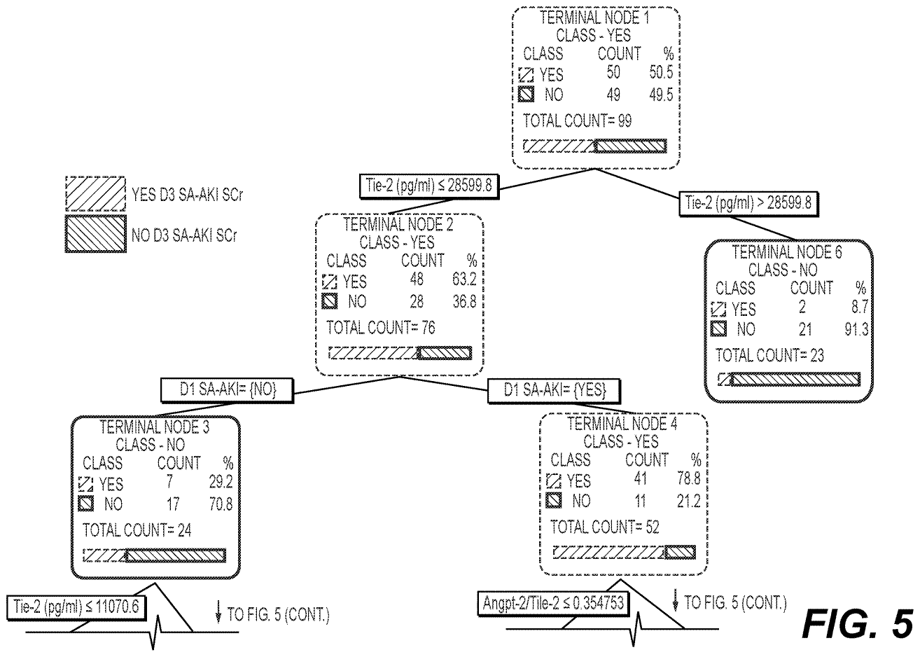

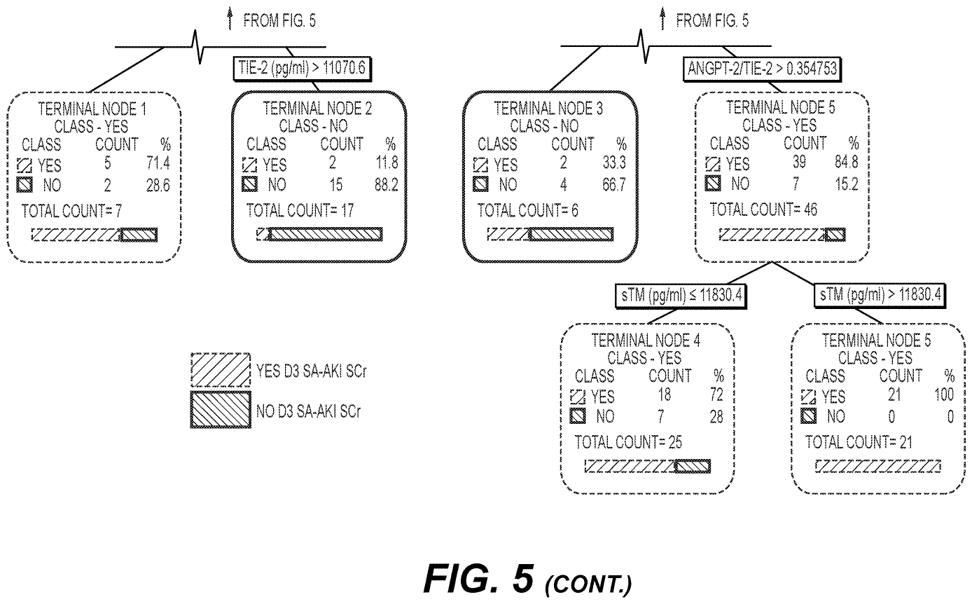

FIG. 5 shows the CART model to predict D3 SA-AKI SCr among patients with high- or intermediate-PERSEVERE-II mortality risk. The root node provides the total number of patients in the derivation cohort, and the number of those with and without D3 SA-AKI SCr, with the respective rates. Each daughter node provides the respective decision rule criterion and the number of those with and without D3 SA-AKI SCr, with the respective rates.

The CART model had 6 terminal nodes (TN) which represent groups of patients who could not be separated further. Terminal nodes (TN) 1, 4, and 5 were deemed to have a high-risk of D3 SA-AKI SCr (≥71.4%); TN2, 3, and 6 were considered to have low-risk of D3 SA-AKI (<11.8%), relative to rate of D3 SA-AKI Scr of 50.5% in the root note. Patients belonging to high or intermediate PERSEVERE-II mortality risk strata who had a Tie-2 concentration >28,599 pg/mL (TN6) concentration had a low risk (8.7%) of D3 SA-AKI SCr. Among those with a Tie-2 concentrations <28,599 pg/mL, those without D1 SA-AKI could be further stratified once again based on Tie-2 concentrations; those with Tie-2 >11,071 pg/mL had low-risk (11.8%) of D3 SA-AKI SCr (TN2) while those with Tie-2≤11,071 pg/mL had high-risk of D3 SA-AKI SCr (71.4%) (TN1). In contrast, those with Tie-2≤28,599 pg/mL and D1 SA-AKI, were further stratified based on Angpt-2/Tie-2 ratios. Patients with Angpt-2/Tie-2<0.35 had low-risk of D3 SA-AKI (33.3%, TN3). Those with high Angpt-2/Tie-2 ratios >0.35 were further stratified based on sTM concentrations; those with sTM ≤11,830 pg/mL were high-risk of D3 SA-AKI SCr (72.0%, TN 4) while those with sTM >11,820 pg/mL had a 100% risk of D3 SA-AKI SCr.

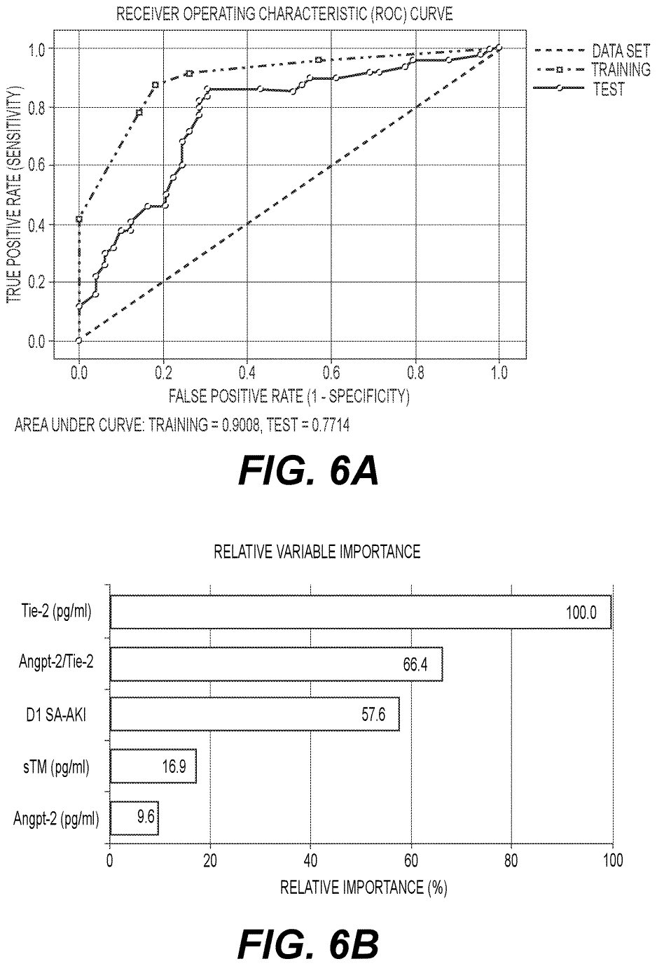

Receiver operating characteristic curve and relative variable importance are shown in FIG. 6. Tie-2 concentration and Angpt-2/Tie-2 ratio were the most important predictor variables in this subset of patients. Terminal nodes (TN) 1, 4, and 5 were deemed to have a high-risk of D3 SA-AKI SCr (>71.4%); TN2, 3, and 6 were considered to have low-risk of D3 SA-AKI SCr (<11.8%). The AUROC for this newly derived “PERSEVERANCE SA-AKI” CART model was 0.90 and 0.77 upon tenfold cross-validation, with a sensitivity of 88% (95% CI 75-95%), specificity of 82% (95% CI 68-91%), positive predictive value of 83% (95% CI 70-91%), and negative predictive value of 87% (95% CI 73-95%) in the derivation cohort.

### Example 6

### Model Performance in the Validation Cohort Demonstrated Reproducibility in Identifying Patients With High Risk of D3 SA-AKI

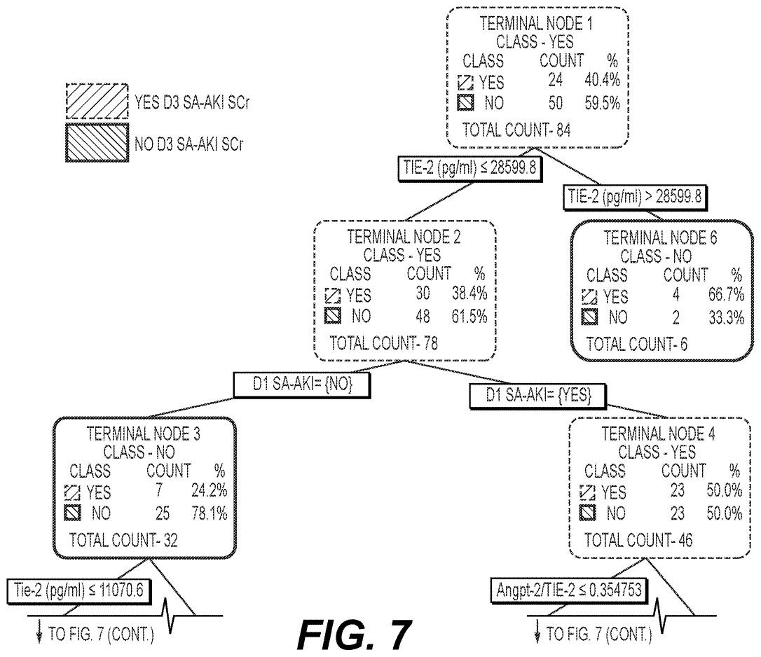

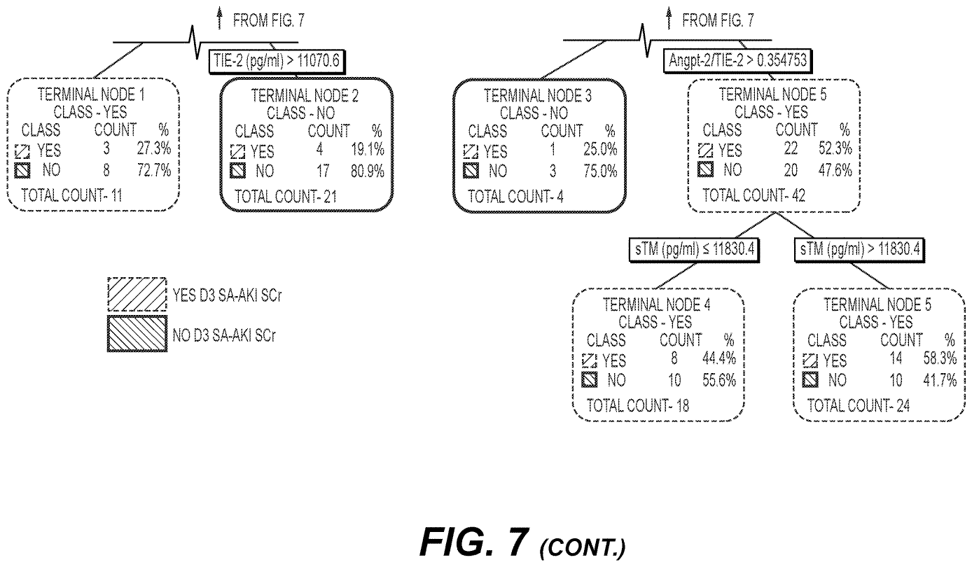

Among the 224 patients in the validation cohort, 29 patients were categorized as high- and 55 patients were categorized as intermediate-PERSEVERE-II mortality risk. Classification of these patients (n=84) in the validation cohort according to the PERSEVERENCE SA-AKI risk model is shown in FIG. 7.

Among patients classified as low D3 SA-AKI risk, 29.0% (9/31) patients actually had D3 SA-AKI SCr. In contrast, among those categorized as high risk, 47.1% (25/53) had D3 SA-AKI.

Finally, considering all 183 patients with high- or intermediate-PERSEVERE-II mortality risk in the derivation and validation cohorts, 64.7% (68/105) and 17.4% (15/78) patients categorized of high- and low-risk, according to the PERSEVERANCE SA-AKI model, actually had D3 SA-AKI SCr, as shown in Table 7.

TABLE 7
Demographic characteristics and clinical outcomes among patients
categorized as high- vs. low D3 SA-AKI risk in the combined
derivation and validation cohorts among those categorized as
having high- or intermediate-PERSEVERE-II mortality risk.

| | High D3 | Low D3 | |
|---|---|---|---|
| | SA-AKI risk | SA-AKI risk | |
| Variable | (n = 105) | (n = 78) | p value |
| Age (Years) | 4 (1,9) | 4(1, 9) | 0.724 |
| Sex, Female (%) | 51 (48.5%) | 29 (37.2%) | 0.193 |
| Race (Self-identified) | | | 0.640 |
| White/Caucasian | 77 (73%) | 57 (73%) | |
| Black/African American | 13 (13%) | 6 (8%) | |
| Other | 14 (13%) | 11 (14%) | |
| Ethnicity | | | 0.114 |
| Hispanic or Latino | 11 (11%) | 14 (11%) | |
| PRISM-III | 16 (10, 24) | 12 (8, 19) | 0.030 |
| 28-day Mortality | 28 (26.7%) | 11 (14.1%) | 0.055 |
| Complicated course | 66 (62.8%) | 26 (33.3%) | <0.001 |
| PICU LOS | 8(4, 15) | 7(3, 15) | 0.085 |
| PICU Free Days | 20 (13, 24) | 21 (13, 25) | 0.086 |
| D3 SA-AKI | 68 (64.7%) | 15(19.2%) | <0.001 |
| Day 7 CRRT* | 25 (33.3%) | 2 (4.6%) | <0.001 |

*Adjusted for study mortality before day 7 and those patients transferred out of ICU without persistent SA-AKI.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the invention. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method of classifying a patient with septic shock as high risk of sepsis-associated acute kidney injury (SA-AKI) or other than high risk of SA-AKI, the method comprising:
receiving a dataset comprising biomarker expression levels of one or more biomarkers selected from the group consisting of: Tie-2, Angpt-2, and sTM, wherein the dataset is obtained from a pediatric patient with septic shock at a first time point;
determining whether the biomarker expression levels of each of the at least one biomarkers are greater than one or more pre-determined cut-off biomarker expression level; and
classifying the patient as high risk of SA-AKI, or other than high risk of SA-AKI, based on the determination of whether the expression levels of each of the at least two biomarkers are greater than the one or more pre-determined cut-off expression level.

2. The method of claim 1, wherein a classification of high risk of SA-AKI comprises:
a) a non-highly elevated level of Tie-2, and absence of day 1 (D1) SA-AKI;
b) a non-highly elevated level of Tie-2, presence of D1 SA-AKI, and an elevated Angpt-2/Tie-2 ratio;
and wherein a classification of other than high risk of SA-AKI comprises:
c) an elevated but non-highly elevated level of Tie-2, and absence of D1 SA-AKI;
d) a non-highly elevated level of Tie-2, presence of day 1 D1 SA-AKI, and a non-elevated Angpt-2/Tie-2 ratio; or
e) a highly elevated level of Tie-2.

3. The method of any preceding claim, wherein biomarker expression levels comprise serum protein biomarker concentrations.

4. The method of any preceding claim, wherein biomarker expression levels are determined by quantifying serum protein biomarker concentrations and/or by cycle threshold (CT) values.

5. The method of any preceding claim, wherein the determined biomarker expression levels comprise expression levels of one or more pairs of biomarkers selected from the group consisting of: Tie-2 and Angpt-2; Tie-2 and sTM; and Angpt-2 and sTM.

6. The method of any preceding claim, wherein the determined biomarker expression levels comprise expression levels of Tie-2, Angpt-2, and sTM.

7. The method of claim 2, wherein biomarker levels are determined by serum protein biomarker concentration, and wherein:
a) an elevated level of Tie-2 corresponds to a serum Tie-2 concentration greater than 11.1 ng/ml;
b) a highly elevated level of Tie-2 corresponds to a serum Tie-2 concentration greater than 28.6 ng/ml;
c) an elevated Angpt-2/Tie-2 ratio corresponds to a ratio greater than 0.354753; and
d) an elevated level of sTM corresponds to a serum sTM concentration greater than 11.8 ng/ml.

8. The method of any preceding claim, wherein the determination of whether the levels of the at least two biomarkers are non-elevated above a cut-off level comprises applying the biomarker expression level data to a decision tree comprising the two or more biomarkers.

9. The method of claim 8, comprising application of the decision tree of FIG. 5.

10. The method of any preceding claim, wherein a classification other than high risk comprises a classification of low risk or intermediate risk.

11. The method of any preceding claim, wherein SA-AKI comprises cardiovascular, respiratory, renal, hepatic, hematologic, and/or neurologic dysfunction, and/or systemic inflammation and/or microvascular endothelial dysfunction, and/or low or no urine output, fluid overload with edema, increased need for supplemental oxygen or intubation and mechanical ventilation, need for dialysis, multi-organ failure and/or death.

12. The method of claim 11, wherein SA-AKI comprises renal dysfunction.

13. The method of claim 11, wherein the patient is undergoing continuous renal replacement therapy (CRRT).

14. The method of any preceding claim, wherein high risk of SA-AKI by day 3 of septic shock or other than high risk of SA-AKI by day 3 of septic shock is determined.

15. The method of any preceding claim, wherein high risk of SA-AKI by day 7 of septic shock or other than high risk of SA-AKI by day 7 of septic shock is determined.

16. The method of any preceding claim, wherein the classification is combined with one or more patient demographic data and/or clinical characteristics and/or results from other tests or indicia of septic shock and/or one or more additional biomarkers and/or platelet count.

17. The method of claim 16, wherein the one or more additional biomarkers is selected from the group consisting of: interleukin-8 (1L-8), heat shock protein 70 kDa 1B (HSPA1B), C—C Chemokine ligand 3 (CCL3), C—C Chemokine ligand 4 (CCL4), Granzyme B (GZMB), Interleukin-1 α (IL-1a), Matrix metallopeptidase 8 (MMP8), Angiopoietin-1 (Angpt-1), Inter-Cellular Adhesion Molecule-1 (ICAM-1), Vascular cell adhesion molecule-1 (VCAM-1), P-selectin, E-selectin, and Platelet and endothelial cell adhesion molecule-1 (PECAM-1).

18. The method of claim 17, wherein the one or more additional biomarkers is selected from the group consisting of: interleukin-8 (IL-8), heat shock protein 70 kDa 1B (HSPA1B), and C—C Chemokine ligand 3 (CCL3).

19. The method of claim 16, wherein the patient demographic data and/or clinical characteristics and/or results from other tests or indicia of septic shock comprise at least one selected from the group consisting of: the septic shock causative organism, the presence or absence or chronic disease, and/or the age, gender, race, ethnicity, and/or co-morbidities of the patient.

20. The method of any preceding claim, wherein the classification is combined with one or more additional population-based risk scores.

21. The method of claim 20, wherein the one or more population-based risk scores comprises at least one selected from the group consisting of: Pediatric Sepsis Biomarker Risk Model (PERSEVERE), Pediatric Sepsis Biomarker Risk Model II (PERSEVERE II), Pediatric Risk of Mortality (PRISM), PRISM III, Pediatric Index of Mortality (PIM), and Pediatric Logistic Organ Dysfunction (PELOD).

22. The method of claim 21, wherein the one or more population-based risk scores comprises PERSEVERE II.

23. The method of any preceding claim, wherein the sample is obtained within the first hour of presentation with septic shock.

24. The method of any preceding claim, wherein the sample is obtained within the first 24 hours of presentation with septic shock.

25. The method of any preceding claim, further comprising administering a treatment comprising one or more high risk therapy to a patient that is classified as high risk, or administering a treatment excluding a high risk therapy to a patient that is not high risk, or to provide a method of treating a pediatric patient with septic shock.

26. The method of claim 25, wherein the one or more high risk therapy comprises at least one selected from the group consisting of: biological and/or immune enhancing therapy, extracorporeal membrane oxygenation/life support, plasmapheresis, pulmonary artery catheterization, high volume continuous hemofiltration, adjuvant hemoperfusion, and/or plasma filtration and/or adsorption therapies.

27. The method of claim 26, wherein the biological and/or immune enhancing therapy comprises administration of recombinant human thrombomodulin, Angiopoietin-2 inhibitors, and/or Tie-2 agonists.

28. The method of any preceding claim, wherein the patient is enrolled in a clinical trial.

29. The method of claim 28, wherein the patient is classified as high risk.

30. The method of claim 29, wherein the method comprises prognostic enrichment through enrollment of the high risk patient in the clinical trial.

31. The method of claim 30, further comprising administering a treatment comprising one or more high risk therapy to the patient in the clinical trial.

32. The method of claim 25, comprising improving an outcome in a pediatric patient with septic shock.

33. The method of claim 25, further comprising:
analyzing a second sample obtained from the treated patient at a second patient, to determine the expression levels of expression levels of one or more biomarkers comprising Tie-2, Angpt-2, and/or sTM;
determining whether the protein biomarker expression levels of each of the biomarkers are greater than one or more pre-determined cut-off protein biomarker expression level;
classifying the patient as high risk of SA-AKI, or other than high risk of SA-AKI, based on the determination of whether the expression levels of each of the biomarkers are greater than the one or more pre-determined cut-off expression level; and
maintaining the treatment being administered if the patient's high risk classification has not changed, or changing the treatment being administered if the patient's high risk classification has changed.

34. The method of claim 33, wherein the second time point is at least 18 hours after the first time point.

35. The method of claim 34, wherein the second time point is in the range of 24 to 96 hours, or longer, after the first time point.

36. The method of claim 34, wherein the second time point is about 1 day, 2 days, 3 days, or longer, after the first time point.

37. The method of claim 36, wherein the second time point is about 2 days after the first time point.

38. The method of claim 37, wherein the first time point is at day 1, wherein day 1 is within 24 hours of a septic shock diagnosis, and the second time point is at day 3.

39. The method of claim 33, wherein a patient classified as high risk after the second time point is administered one or more high risk therapy.

40. The method of claim 39, wherein the one or more high risk therapy comprises at least one selected from the group consisting of: biological and/or immune enhancing therapy, extracorporeal membrane oxygenation/life support, plasmapheresis, pulmonary artery catheterization, high volume continuous hemofiltration, adjuvant hemoperfusion, and/or plasma filtration and/or adsorption therapies.

41. The method of claim 40, wherein the one or more high risk therapy comprises a biological and/or immune enhancing therapy.

42. The method of claim 33, wherein a patient not classified as high risk after the second time point is administered a treatment excluding a high risk therapy.

43. The method of claim 33, wherein the patient classified as high risk and administered one or more high risk therapy after the first time point is not classified as high risk after the second time point.

44. The method of any preceding claim, wherein one or more biomarker cut-off level is determined by one or more trained machine learning models based on a dataset generated from a cohort of pediatric patients with and without SA-AKI.

45. The method of any preceding claim, wherein the data from the cohort of pediatric patients with and without SA-AKI is provided to one or more machine learning models as input, and wherein the one or more trained machine learning model is based on a dataset generated from the biomarker cutoff levels in the patients of the cohort.

46. The method of any preceding claim, wherein one or more biomarker cut-off level is determined by a trained machine learning model, and wherein one or more machine learning models is used to classify the patient as high risk of SA-AKI, or other than high risk of SA-AKI.

47. The method of any preceding claim, as part of a companion diagnostic or a point of care device or kit.

48. A diagnostic kit, test, or array comprising a reporter hybridization probe, and a capture hybridization probe specific for each of two or more mRNA, DNA, or protein biomarkers selected from the group consisting of: Tie-2, Angpt-2, and sTM.

49. The diagnostic kit, test, or array of claim 48, wherein the biomarkers comprise Tie-2, Angpt-2, and sTM.

50. The diagnostic kit, test, or array of claim 48, wherein the biomarkers further comprise one or more of interleukin-8 (IL-8), heat shock protein 70 kDa 1B (HSPA1B), C—C Chemokine ligand 3 (CCL3), C—C Chemokine ligand 4 (CCL4), Granzyme B (GZMB), Interleukin-1 α (IL-1a), and/or Matrix metallopeptidase 8 (MMP8).

51. The diagnostic kit, test, or array of claim 48, further comprising a collection cartridge for immobilization of the hybridization probes.

52. The diagnostic kit, test, or array of claim 48, wherein the reporter and the capture hybridization probes comprise signal and barcode elements, respectively.

53. An apparatus or processing device suitable for detecting two or more biomarkers selected from the group consisting of: Tie-2, Angpt-2, and sTM.

54. The apparatus or processing device of claim 53, wherein the biomarkers comprise Tie-2, Angpt-2, and sTM.

55. The apparatus or processing device of claim 54, wherein the biomarkers further comprise one or more of interleukin-8 (IL-8), heat shock protein 70 kDa 1B (HSPA1B), C—C Chemokine ligand 3 (CCL3), C—C Chemokine ligand 4 (CCL4), Granzyme B (GZMB), Interleukin-1 α (IL-1a), and/or Matrix metallopeptidase 8 (MMP8).

56. A composition comprising a reporter hybridization probe, and a capture hybridization probe specific for each of two or more biomarkers selected from the group consisting of: Tie-2, Angpt-2, and sTM.

57. The composition of claim 56, wherein the biomarkers comprise Tie-2, Angpt-2, and sTM.

58. The composition of claim 56, wherein the biomarkers further comprise one or more of interleukin-8 (IL-8), heat shock protein 70 kDa 1B (HSPAlB), C—C Chemokine ligand 3 (CCL3), C—C Chemokine ligand 4 (CCL4), Granzyme B (GZMB), Interleukin-1 α (IL-1a), and/or Matrix metallopeptidase 8 (MMP8).
